(19) United States
(12) Patent Application Publication
(10) Pub. No.: US 2025/0213717 A1
(43) Pub. Date: Jul. 3, 2025

(54) HYDROGEL FORMULATIONS FOR VLP THERAPEUTICS

(71) Applicant: THE REGENTS OF THE UNIVERSITY OF CALIFORNIA, Oakland, CA (US)

(72) Inventors: Nicole F. STEINMETZ, San Diego, CA (US); Christian NKANGA, San Diego, CA (US)

(21) Appl. No.: 18/850,029

(22) PCT Filed: Mar. 23, 2023
(86) PCT No.: PCT/US2023/016038

§ 371 Date: Sep. 23, 2024

Related U.S. Application Data
(60) Provisional application No. 63/323,443, filed on Mar. 24, 2022.

(51) Int. Cl. A61K 47/69; A61K 47/54; A61K 47/61; A61P 35/00

(52) U.S. Cl. CPC A61K 47/6901; A61K 47/548; A61K 47/61; A61P 35/00

(57) ABSTRACT

Formulations for improved delivery of therapeutic virus-like particles containing therapeutic peptides are provided. Method for making the formulations and use thereof, are further provided.

### CROSS-REFERENCE TO RELATED APPLICATION

This application claims priority under 35 U.S.C. § 119(e) to U.S. Provisional Application No. 63/323,443, filed Mar. 24, 2022, the contents of which are incorporated herein by reference in its entireties.

### STATEMENT OF GOVERNMENT SUPPORT

This invention was made with government support under CA253615 awarded by the National Institutes of Health, and under CHE2116298 awarded by the National Science Foundation. The government has certain rights in the invention.

### INCORPORATION BY REFERENCE

All publications, patents, and patent applications mentioned in this specification and attached Appendices are herein incorporated by reference to the same extent as if each individual publication, patent, patent application or appendix, was specifically and individually indicated to be incorporated by reference.

### BACKGROUND

Cowpea mosaic virus (CPMV) is a potent adjuvant for vaccines and cancer immunotherapy, but for either of these applications, repeat administration is needed to achieve potent efficacy. Therefore, state of the art slow-release formulations are needed. Earlier versions have had limited success due to technical problems. This disclosure satisfies this need and provides related advantages as well.

This disclosure satisfies this need and provides related advantages as well.

### SUMMARY OF THE DISCLOSURE

Applicant provides compositions containing CPMV nanoparticles that are effectively formulated in chitosan/GP hydrogels and are released over several months as intact and biologically active particles with conserved immunotherapeutic efficacy. The disclosed formulations not only represent a single-dose vaccine candidate to address future pandemics, but also facilitate the development of long-lasting plant virus-based nanomedicines for diseases that require long-term treatment.

Provided herein are formulations comprising, or consisting of, or consisting essentially of, a virus-like particle (VLP) derived from a plant virus, a chitosan polymer and β-glycerophosphate (GP). Also provided are formulations comprising, or consisting of, or consisting essentially of, a virus-like particle (VLP) derived from a plant virus conjugated to a therapeutic peptide, a chitosan polymer and β-glycerophosphate (GP). Non-limiting examples of chitosans include those having a molecular weight from about 250 kDa to about 1500 kDa. In a further aspect, the concentration of the VLP in the formulation comprises from about 0.1 mg/ml to about 7 mg/ml at 37° C.

The VLP in the formulation are from a plant virus from the group of the genus Bromovirus, Comovirus, or Tymovirus. Non-limiting examples of such include a plant virus selected from Cowpea chlorotic mottle virus (CCMV), Cowpea mosaic virus (CPMV), or Physalis mottle virus (PhMV).

In another aspect, the VLP comprises a capsid protein, optionally a modified capsid protein, further optionally from CCMV, CPMV, PhMV, or a combination thereof.

When the formulation comprises, or consists of, or consists essentially of, a therapeutic peptide, the therapeutic peptide comprises an extracellular or transmembrane polypeptide that specifically binds to a receptor expressed on a target cell, optionally an immune cell. The immune cell can be an antigen presenting cell, a macrophage, B cell, a dendritic cell, or a nature killer (NK) cell. In one aspect, the macrophage is a tumor-associated macrophage (TAM). Alternatively, the macrophage, optionally TAM, is located within a tumor microenvironment (TME).

In another aspect, the therapeutic peptide comprises an antibody or antigen binding fragment thereof. In one aspect, the antigen or antigen binding fragment is a cancer antigen or fragment thereof. In another aspect the antigen or antigen binding fragment is a B cell antigen as described herein. The formulations further contain an additional therapeutic agent encapsulated within the VLP, optionally a cancer therapy. In one aspect, the antigen or antigen binding fragment is a cancer antigen or fragment thereof and the formulations further contain an additional therapeutic agent encapsulated within the VLP, optionally a cancer therapy

In another aspect, the therapeutic peptide comprises, or consists of, or consists essentially of, a peptide that induces an immune response against a pathogen, e.g., when the pathogen is a coronavirus, e.g., SARS-CoV-2. In another aspect, the therapeutic protein comprises a B-cell epitope selected from amino acids 553-570, 625-636 or 809-826.

In another aspect, the therapeutic peptide is conjugated to the VLP by a linker, optionally by conjugating the VLP lysine residues to the N-hydroxysuccinimide (NHS)-activated ester of Cy5.

In one particular embodiment, the therapeutic protein comprises the B-cell epitope comprising amino acids 809-826 optionally conjugated to the VLP lysine residues to the N-hydroxysuccinimide (NHS)-activated ester of Cy5.

In another embodiment, the formulation contains a plurality of VLPs. The plurality can of the VLPs and/or the therapeutic peptides are the same or different from each other.

Also provided are compositions comprising one or more formulations as described herein, and a carrier, optionally a pharmaceutically acceptable carrier. In one aspect, the composition is formulated for in vitro or in vivo use, optionally systemic administration. In one embodiment, the composition is formulated for local administration. In another embodiment, the composition is formulated for parenteral administration, optionally for intravenous, subcutaneous, intramuscular, intracerebral, intranasal, intra-arterial, intra-articular, intradermal, intravitreal, intraosseous infusion, intraperitoneal, or intrathecal administration.

In one aspect, the composition further comprises a preservative or stabilizer, that can be lyophilized or frozen.

The compositions can be used for treating a disease or condition or inducing an immune response in a subject in need thereof, comprising administering to the subject a formulation or composition as described above and herein. Non-limiting examples of such include cancer (e.g., metastatic or primary, e.g., colon cancer), an inflammatory condition, an autoimmune disease, an allergy, or a pathogenic infection. In one aspect, the therapeutic peptide induces an immune response to induce an immune response for the treating or preventing a COVID infection. In another aspect, the formulation does not comprise a therapeutic peptide and the disease is cancer, e.g., colon cancer.

Also provided are methods for formulating a VLP, comprising admixing a virus-like particle (VLP) derived from a plant virus that is optionally conjugated to a therapeutic peptide, a chitosan polymer and β-glycerophosphate (GP).

Non-limiting examples of chitosans include those having a molecular weight from about 250 kDa to about 1500 kDa. In a further aspect, the concentration of the VLP in the formulation comprises from about 0.1 mg/ml to about 7 mg/ml at 37° C.

The VLP in the method are from a plant virus from the group of the genus Bromovirus, Comovirus, or Tymovirus. Non-limiting examples of such include a plant virus selected from Cowpea chlorotic mottle virus (CCMV), Cowpea mosaic virus (CPMV), or Physalis mottle virus (PhMV). In another aspect, the VLP comprises a capsid protein, optionally a modified capsid protein, further optionally from CCMV, CPMV, PhMV, or a combination thereof.

The therapeutic peptide of the method can comprise an extracellular or transmembrane polypeptide that specifically binds to a receptor expressed on a target cell, optionally an immune cell. The immune cell can be an antigen presenting cell, a macrophage, B cell, a dendritic cell, or a nature killer (NK) cell. In one aspect, the macrophage is a tumor-associated macrophage (TAM). Alternatively, the macrophage, optionally TAM, is located within a tumor microenvironment (TME).

In another aspect, the therapeutic peptide comprises an antibody or antigen binding fragment thereof. The method can further admix an additional therapeutic agent encapsulated within the VLP, optionally a cancer therapy.

In another aspect, the therapeutic peptide comprises a peptide that induces an immune response against a pathogen, e.g., when the pathogen is a coronavirus, e.g., SARS-CoV-2. In another aspect, the therapeutic protein comprises a B-cell epitope selected from amino acids 553-570, 625-636 or 809-826.

In another aspect, the method further comprises admixing a therapeutic peptide conjugated to the VLP by a linker, optionally by conjugating the VLP lysine residues to the N-hydroxysuccinimide (NHS)-activated ester of Cy5.

In one particular embodiment, a therapeutic protein that is added comprises the B-cell epitope comprising amino acids 809-826 optionally conjugated to the VLP lysine residues to the N-hydroxysuccinimide (NHS)-activated ester of Cy5.

In another embodiment, the method admixes a plurality of VLPs. The plurality can of the VLPs and/or the therapeutic peptides are the same or different from each other.

The method further comprises preparing compositions by admixes one or more formulations as described herein, with a carrier, optionally a pharmaceutically acceptable carrier.

In one aspect, the method further admixing a preservative or stabilizer. The method further comprises lyophilizing or freezing the formulation or composition.

Further provide are kits comprising the formulations or the compositions as described herein, and instructions for use.

### BRIEF DESCRIPTION OF THE DRAWING

FIGS. 1A-1E: Characterization of CPMV and Cy5-CPMV. (FIG. 1A) Bioconjugation reaction, labeling of CPMV with sulfo-cyanine 5 (Cy5) using NHS chemistry. Black dots on the CPMV surface represent lysine residues. (FIG. 1B) SDS-PAGE comparing CPMV wild-type and Cy5-conjugated CPs, demonstrating similar electrophoretic profiles and thus successful covalent attachment. (FIG. 1C) Native agarose gel electrophoresis demonstrating the similar electrophoretic mobility of CPMV/Cy5-CPMV (viral proteins, RNA and Cy5 fluorophore), suggesting the particles are intact. (FIG. 1D) Dynamic light scattering, indicating the nanoparticulate nature of CPMV/Cy5-CPMV samples. (FIG. 1E) Size exclusion chromatography, confirming CPMV/Cy5-CPMV particle integrity by the co-elution of all viral components in the same peak. The dashed curve represents viral CP absorbance at λ=260 nm, the solid curve is the RNA signal at λ=280 nm and the solid line closest to the X-axis is Cy5 detected at λ=647 nm.

FIGS. 2A-2G: Preparation and characterization of hydrogels. (FIG. 2A) CPMV particles were dispersed in chitosan/GP hydrogels. (FIG. 2B) Design-of-experiment plots (from Minitab software) showing the impact of two formulation variables (chitosan molecular weight and CPMV concentration) on gelation time. (FIG. 2C) Rheological properties of liquid formulations, showing variations in relative viscosity at 25° C. F1 is the thick dotted line, F2 is the thin dotted line and F3 is the solid line. (FIG. 2D) Gel height variations measured at different time points following hydrogel incubation in PBS at 37° C. (n=3). (FIG. 2E) The experimental setting used for in vitro gel swelling/degradation and release analysis, showing the homogeneous dispersion of Cy5-CPMV in hydrogel F3 versus PBS. (FIG. 2F) Full data set showing in vitro Cy5-CPMV release from hydrogels versus soluble Cy5-CPMV/PBS at 37° C. (n=3). (FIG. 2G) Release data excerpt showing the difference between the three hydrogel formulations. Asterisks indicate significant differences between groups (*p<0.05; **p<0.01).

FIG. 3: TEM images of Cy5-CPMV released in vitro from hydrogels following incubation in PBS for 14 days, confirming the integrity and stability of Cy5-CPMV particles within the hydrogel matrix.

FIGS. 4A-4C: In vivo retention/release of Cy5-CPMV from hydrogels (F1, F2 and F3) versus soluble Cy5-CPMV. (FIG. 4A) Fluorescence images and (FIG. 4B) fluorescence intensity at the injection site in female BALB/c mice (n=5 per group) following a single subcutaneous injection of F1, F2 or F3 (450 g Cy5-CPMV) or soluble Cy5-CPMV (450 g) on day 0. Asterisks indicate significant differences between F3 and Cy5-CPMV (*p<0.05). (FIG. 4C) Comparing local retention of a single subcutaneous dose of hydrogel F3 (containing 200 g Cy5-CPMV) versus two doses of soluble Cy5-CPMV (100 g injected at days 0 and 14) in female BALB/c mice. Fluorescence images, demonstrating extended tissue residence of the F3 hydrogel compared to soluble Cy5-CPMV.

FIGS. 5A-5D: Conjugation of the B-cell peptide epitope 826 to CPMV with a CGGG linker. (FIG. 5A) The two-step synthesis of 826-CPMV conjugates. (FIG. 5B) SDS-PAGE analysis comparing the coat proteins (CP) from wild-type and modified CPMV particles. (FIG. 5C) Agarose gel showing the co-localization of viral RNA (under UV light) with CP (revealed by staining with Coomassie Brilliant Blue). (FIG. 5D) TEM images confirming particle integrity following the bioconjugation reaction. Scale bar=100 nm.

FIGS. 6A-6D: Antibody response following the immunization of BALB/c mice (n=4 per group). (FIG. 6A) Mice were subcutaneously (S.C.) injected once with hydrogel F3 (containing 200 g 826-CPMV) or 200 g of soluble 826-CPMV in PBS, or with 2×100 g soluble 826-CPMV in PBS as a prime-boost regimen. Blood samples were withdrawn by retro-orbital bleeding according to the schedule as shown. (FIG. 6B) ELISA to detect IgG (from immunized mouse serum) binding to epitope 826. (FIG. 6C) ELISA data curves showing IgG titers of immunized mice against epitope 826 from weeks 2 to 20. (FIG. 6D) Longitudinal IgG titers over 20 weeks; indicating that F3 group continuously differed from the control blank group to much greater extent than soluble particle (with p values included for weeks 16 and 20 to show the differences). Asterisks indicate significant differences between a study group and control blank group (*p<0.05; **p<0.01; ***p<0.001; ****p<0.0001); with green color (also shown as “-●-”) referring to soluble 826 CPMV 100 (×2) group, blue to 826 CPMV 200 (also shown as “-▪-”) group and red (also shown as “-▴-”) to F3 group.

FIGS. 7A-7B: Antibody isotyping using mouse sera from weeks 4 and 12 (n=4 per group). (FIG. 7A) Immunoglobulin isotypes and IgG subclasses, showing comparable antibody profiles at week 4, but enhanced IgG1 production by the F3 group at week 12 (three arrows). (FIG. 7B) IgG profiling expressed as the IgG1/IgG2a ratio, demonstrating a Th1-biased response (IgG1/IgG2a ratio <1) for all groups at week 4, but a remarkable shift to a Th2-biased response (IgG1/IgG2a ratio >1) exclusively in the F3 group.

FIG. 8: Schematic presentation of CPMV formulation in chitosan/GP hydrogels. Images of inverted Eppendorf tubes illustrate the no flow behavior and increased turbidity occurring when gel forms upon heating.

FIGS. 9A-9B: (FIG. 9A) Fluorescence intensity longitudinally determined using ROI analysis of mice IP space (n=5) images, tracking the persistence of fluorescence signals over 21 days following IP single injection of 450 g Cy5-CPMV in PBS (Cy5-CPMV group), or 450 g Cy5-CPMV formulated in hydrogels (F1, F2, and F3); the control group was composed of untreated mice. (FIG. 9B) Fluorescence intensity measured by ROI analysis of organs (n=5) collected at the study endpoint (day 21). The connotation “ns” stands for not significantly different from the control/untreated group. From left to right, control, F1, F2, F3 and then Cy5-CPMV. Asterisks indicate statistical differences versus the control (*p<0.05; **p<0.01; ****p<0.0001); hashtags show significant differences between a given hydrogel formulation and soluble Cy5-CPMV (#p<0.05; ##p<0.01; ####p<0.0001).

FIGS. 10A-10D: In situ vaccination with CPMV-in-chitosan/GP hydrogel inhibits colon cancer growth in the intraperitoneal space (n=5 mice per group). (FIG. 10A) Study design: BALB/c mice were inoculated intraperitoneally with 1 million of luciferase positive CT26 cells, and the following treatments were immediately I.P. injected once with hydrogel F3 (containing 200 g CPMV) only or hydrogel F3+100 g soluble CPMV in PBS, or 200 g of soluble CPMV in PBS, or twice with 100 g soluble CPMV in PBS as a prime-boost regimen. Cancer cell growth was longitudinally assessed by bioluminescence imaging (FIG. 10B) and intensity measurements (FIG. 10C) in the intraperitoneal space 5 min following I.P. injection of luciferin 15 mg mL⁻¹/150 μL. The luminescence was calculated using ROI analysis from the Living Image 3.0 software. Asterisks indicate statistical differences versus the control (*p<0.05; **p<0.01); hashtags show significant differences between a given hydrogel treatment and soluble prime-boost CPMV particle (#p<0.05; ##p<0.01). Tumor burden and ascites development were monitored by measuring abdominal circumferences (FIG. 10D). The p values indicate the difference with the control group (blank F3).

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the term “comprising” is intended to mean that the methods include the recited steps or elements, but do not exclude others. “Consisting essentially of” shall mean rendering the claims open only for the inclusion of steps or elements, which do not materially affect the basic and novel characteristics of the claimed methods. “Consisting of” shall mean excluding any element or step not specified in the claim. Embodiments defined by each of these transition terms are within the scope of this disclosure.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the terms “treating,” “treatment” and the like mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder. In one aspect, the term “treatment” excludes prophylaxis.

As used herein, to “treat” further includes systemic amelioration of the symptoms associated with the pathology and/or a delay in onset of symptoms. Clinical and sub-clinical evidence of “treatment” will vary with the pathology, the individual and the treatment. In one aspect, treatment excludes prophylaxis.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disease or condition, such as one or more adverse symptoms, disorders, illnesses, pathologies, diseases, or complications caused by or associated with the disease or condition, or an improvement in an underlying cause or a consequence of the disease or condition, or a reversal of the disease or condition.

Treatment can therefore result in decreasing, reducing, inhibiting, suppressing, limiting, controlling or preventing a disease or condition, or an associated symptom or consequence, or underlying cause; decreasing, reducing, inhibiting, suppressing, limiting, controlling or preventing a progression or worsening of a disease, condition, symptom or consequence, or underlying cause; or further deterioration or occurrence of one or more additional symptoms of the disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disease or symptom in the subject, such as one or more adverse symptoms, disorders, illnesses, pathologies, diseases, or complications caused by or associated with a disease or condition. Treatment methods affecting one or more underlying causes of the condition, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the disease or condition, over a short or long duration of time (hours, days, weeks, months, etc.). In one aspect, prophylaxis or prevention is excluded from “treatment” or “therapeutic benefit.”

As used herein, the disease or condition comprise, or consists essentially of, or yet further consists of, a cancer, e.g., a solid tumor or a hematologic malignancy. Exemplary solid tumors include, but are not limited to, bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, or stomach cancer. Exemplary hematologic malignancy include, but are not limited to, chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

As used herein, the term “an equivalent thereof” when referring to sequence identity comprises at least 80%, 90%, 95%, 96%, 97%, 98%, or 99% sequence identify to the respective reference sequence of which it is compared to, while still retaining a functional activity. In some instances, a functional activity refers to the modulation of an immunostimulatory effect on an immune cell.

As used herein the B cell epitopes from SARS-CoV-2 protein follow conventional nomenclature, for example epitope 826 comprises, or consists of, or consists essentially of PSKPSKRSFIEDLLFNKV. Additional B cell epitopes are provided below**:

| S domain | location (name) | sequence |
|---|---|---|
| S1 | 369-386 | YNSASFSTFKCYGVSPTK |
| S2 | 806-820 | LPDPSKPSKRSFIED |
| S1 | 456-460 | FRKSN |
| S2 | 809-826 | PSKPSKRSFIEDLLFNKV |
| S1 | 553-570 | TESNKKFLPFQQFGRDIA |
| S1 | 553-564 | TESNKKFLPFQQ |
| S1 | 625-636 | HADQLTPTWRVY |
| S2 | 1148-1159 | FKEELDKYFKNH |
| S1 | 92-106 | FASTEKSNIIRGWIF |
| S1 | 139-153 | PFLGVYYHKNNKSWM |
| S1 | 406-420 | EVRQIAPGQTGKIAD |
| S1 | 439-454 | NNLDSKVGGNYNYLYR |
| S1 | 455-469 | LFRKSNLKPFERDIS |

**Reproduced from Table 1 of Ortega-Rivera et al. (2021), J. Am. Chem. Soc., Vol. 143:1478-14765, incorporated herein by reference.

### Virus-Like Particles (VLPs)

As utilized herein in the formulations of this disclosure, a VLP is a non-native VLP that comprise, or consists essentially of, or yet further consists of, one or more viral particles, e.g., a capsid, derived from a plant virus. In some instances, the plant virus is from the genus Bromovirus, Comovirus, Tymovirus, or Sobemovirus. In some cases, the VLP is derived from Cowpea chlorotic mottle virus (CCMV), Cowpea mosaic virus (CPMV), Physalis mottle virus (PhMV), or Sesbania mosaic virus (SeMV).

In some instances, the VLP comprise, or consists essentially of, or yet further consists of, a capsid protein derived from a plant virus. In some instances, the capsid protein is a wild-type protein derived from the plant virus. In other instances, the capsid protein is a variant of the wild-type protein derived from the plant virus. In additional instances, the capsid protein is a modified protein, either full-length or truncated version.

As used herein, the term “Virus-like particle” or “VLP” refers to a non-replicating, viral shell, derived from one or more viruses (e.g., one or more plant viruses described herein). VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. VLPs can also be engineered, e.g., comprising, or consisting essentially of, or yet further consisting of, one or more viral proteins that comprise, or consists essentially of, or yet further consists of, a modification. Methods for producing VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354: 53073, 2012).

In some embodiments, the VLP is derived from Cowpea chlorotic mottle virus (CCMV). CCMV is a spherical plant virus that belongs to the Bromovirus genus. Several strains have been identified and include, but not limited to, Car1 (Ali, et al., 2007. J. Virological Methods 141:84-86), Car2 (Ali, et al., 2007. J. Virological Methods 141:84-86, 2007), type T (Kuhn, 1964. Phytopathology 54:1441-1442), soybean (S) (Kuhn, 1968. Phytopathology 58:1441-1442), mild (M) (Kuhn, 1979. Phytopathology 69:621-624), Arkansas (A) (Fulton, et al., 1975. Phytopathology 65: 741-742), bean yellow stipple (BYS) (Fulton, et al., 1975. Phytopathology 65: 741-742), R (Sinclair, ed. 1982. Compendium of Soybean Diseases. 2^{nd} ed. The American Phytopathological Society, St. Paul. 104 pp.), and PSM (Paguio, et al., 1988. Plant Diseases 72(9): 768-770).

In some instances, the VLP from CCMV comprise, or consists essentially of, or yet further consists of, a plurality of capsid proteins. In some instances, the capsid protein is a wild-type CCMV capsid, optionally expressed by Car1, Car2, type T, soybean (S), mild (M), Arkansas (A), bean yellow stipple (BYS), R, or PSM strain. In other instances, the capsid protein is a modified capsid protein, e.g., comprising, or consisting essentially of, or yet further consisting of, one or more substitutions, insertions, and/or deletions. In some cases, the CCMV capsid comprise, or consists essentially of, or yet further consists of, the sequence as set forth in the UniProtKB ID P03601:

(SEQ ID NO: )
MSTVGTGKLTRAQRRAAARKNKRNTRVVQPVIVEPIASGQGKAIKAWTG
YSVSKWTASCAAAEAKVTSAITISLPNELSSERNKQLKVGRVLLWLGLL
PSVSGTVKSCVTETQTTAAASFQVALAVADNSKDVVAAMYPEAFKGITL
EQLTADLTIYLYSSAALTEGDVIVHLEVEHVRPTFDDSFTPVY,
or
an equivalent thereof.

In some cases, the VLP from CCMV is prepared by the method as described in Ali et al., “Rapid and efficient purification of Cowpea chlorotic mottle virus by sucrose cushion ultracentrifugation,” Journal of Virological Methods 141: 84-86 (2007).

In some embodiments, the VLP is derived from Cowpea mosaic virus (CPMV). CPMV is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1).

In some instances, the VLP from CPMV comprise, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprise, or consists essentially of, or yet further consists of, the sequence as set forth in the UniProtKB ID P03599 (residues 460-833):

(SEQ ID NO: )
MEQNLFALSLDDTSSVRGSLLDTKFAQTRVLLSKAMAGGDVLLDEYLYD
VVNGQDFRATVAFLRTHVITGKIKVTATTNISDNSGCCLMLAINSGVRG
KYSTDVYTICSQDSMTWNPGCKKNFSFTFNPNPCGDSWSAEMISRSRVR
MTVICVSGWTLSPTTDVIAKLDWSIVNEKCEPTIYHLADCQNWLPLNRW
MGKLTFPQGVTSEVRRMPLSIGGGAGATQAFLANMPNSWISMWRYFRGE
LHFEVTKMSSPYIKATVTFLIAFGNLSDAFGFYESFPHRIVQFAEVEEK
CTLVFSQQEFVTAWSTQVNPRTTLEADGCPYLYAIIHDSTTGTISGDFN
LGVKLVGIKDFCGIGSNPGIDGSRLLGAIAQ,
or
an equivalent thereof.

In some cases, the mature small capsid protein is a wild-type mature small capsid protein, optionally expressed by SB or Vu strain. In other instances, the mature small capsid protein is a modified mature small capsid protein, e.g., comprising, or consisting essentially of, or yet further consisting of, one or more substitutions, insertions, and/or deletions. In some cases, the mature small capsid protein comprise, or consists essentially of, or yet further consists of, s the sequence as set forth in the UniProtKB ID P03599 (residues 834-1022):

(SEQ ID NO: )
GPVCAEASDVYSPCMIASTPPAPFSDVTAVTFDLINGKITPVGDDNWNT
HIYNPPIMNVLRTAAWKSGTIHVQLNVRGAGVKRADWDGQVFVYLRQSM
QPGSAMLNFSFDIIGPNSGFEFAESPWANQTTWYLECVATNPRQIQQFE
VNPESYDARTFVISNMRFDPNFRVAGNILMPPFPLSTETPPL,
or
an equivalent thereof.

In some embodiments, the VLP is derived from Physalis mottle virus (PhMV). PhMV is a single stranded RNA virus that belongs to the genus Tymovirus. In some instances, the VLP from PhMV comprises, or consists essentially of, or yet further consists of, a plurality of coat proteins. In some instances, the coat protein is a wild-type PhMV coat protein. In other instances, the coat protein is a modified coat protein, e.g., comprising, or consisting essentially of, or yet further consisting of, one or more substitutions, insertions, and/or deletions. In some cases, the PhMV coat comprise, or consists essentially of, or yet further consists of, s the sequence as set forth in the UniProtKB ID P36351:

(SEQ ID NO: )
MDSSEVVKVKQASIPAPGSILSQPNTEQSPAIVLPFQFEATTFGTAETA
AQVSLQTADPITKLTAPYRHAQIVECKAILTPTDLAVSNPLTVYLAWVP
ANSPATPTQILRVYGGQSFVLGGAISAAKTIEVPLNLDSVNRMLKDSVT
YTDTPKLLAYSRAPTNPSKIPTASIQISGRIRLSKPMLIAN,
or
an equivalent thereof.

In some embodiments, the VLP is derived from Sesbania mosaic virus (SeMV). SeMV is a positive stranded RNA virus that belongs to the genus Sobemovirus. In some instances, the VLP from SeMV comprise, or consists essentially of, or yet further consists of, a plurality of capsid proteins. In some instances, the capsid protein is a wild-type SeMV capsid protein. In other instances, the capsid protein is a modified capsid protein, e.g., comprising, or consisting essentially of, or yet further consisting of, one or more substitutions, insertions, and/or deletions. In some cases, the SeMV capsid comprise, or consists essentially of, or yet further consists of, s the sequence as set forth in the UniProtKB ID Q9EB06:

(SEQ ID NO: )
MAKRLSKQQLAKAIANTLETPPQPKAGRRRNRRRQRSAVQQLQPTQAGI
SMAPSAQGAMVRIRNPAVSSSRGGITVLTHSELSAEIGVTDSIVVSSEL
VMPYTVGTWLRGVAANWSKYSWLSVRYTYIPSCPSSTAGSIHMGFQYDM
ADTVPVSVNQLSNLRGYVSGQVWSGSAGLCFINGTRCSDTSTAISTTLD
VSKLGKKWYPYKTSADYATAVGVDVNIATPLVPARLVIALLDGSSSTAV
AAGRIYCTYTIQMIEPTASALNN,
or
an equivalent thereof.

As used herein, the term “an equivalent thereof” in reference to a polynucleotide or a protein (e.g., a capsid or coat protein) include a polynucleotide or a protein that comprise, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a VLP.

As used herein, the term “modification” include, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a capsid derived from the plant virus. In some instances, a capsid described herein includes, e.g., a modified capsid comprising, or consisting essentially of, or yet further consisting of, at least 70%, 80%, 85%, 90%, 95%, 96%, 97%, 98%, 99%, or 100% sequence identity to its respective wild-type version.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nim.nih.gov/blast/Blast.cgi.

Modified capsid polypeptides include, for example, non-conservative and conservative substitutions of the capsid amino acid sequences.

As used herein, the term “conservative substitution” denotes the replacement of an amino acid residue by another, chemically or biologically similar residue. Biologically similar means that the substitution does not destroy a biological activity or function, e.g., assembly of a viral capsid.

Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

In some instances, a VLP described herein further comprise, or consists essentially of, or yet further consists of, a label or a tag, e.g., such as a detectable label. A detectable label can be attached to, e.g., to the surface of a VLP.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As, ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Fe(III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the VLP. In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

Also provided herein is the VLP as described herein further comprising, or consisting essentially of, or yet further consisting of an additional therapeutic agent.

In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the VLP with or without the additional therapeutic agent comprise, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprise, or consists essentially of, or yet further consists of, s chemotherapy. In other cases, the first-line treatment comprise, or consists essentially of, or yet further consists of, s radiation therapy. A skilled artisan would readily understand that different first-line treatments may be applicable to different type of cancers.

In some cases, the additional therapeutic agent comprise, or consists essentially of, or yet further consists of, or is used as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprise, or consists essentially of, or yet further consists of, a salvage therapy.

In some cases, the additional therapeutic agent comprise, or consists essentially of, or yet further consists of, a palliative therapy.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, s an immune checkpoint inhibitor. Exemplary checkpoint inhibitors include:
PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736;
PD-L2 inhibitors such as GlaxoSmithKline's AMP-224 (Amplimmune), and rHIgM12B7;
PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd;
CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam;
LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12;
B7-H3 inhibitors such as MGA271;
KIR inhibitors such as Lirilumab (IPH2101);
CD137 inhibitors such as urelumab (BMS-663513, Bristol-Myers Squibb), PF-05082566 (anti-4-1BB, PF-2566, Pfizer), or XmAb-5592 (Xencor);
PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprise, or consists essentially of, or yet further consists of, s pembrolizumab, nivolumab, tremelimumab, or ipilimumab.

In some cases, the additional therapeutic agent comprise, or consists essentially of, or yet further consists of, s an antibody such as alemtuzumab, trastuzumab, ibritumomab tiuxetan, brentuximab vedotin, ado-trastuzumab emtansine, or blinatumomab.

In some cases, the additional therapeutic agent comprise, or consists essentially of, or yet further consists of, a cytokine. Exemplary cytokines include, but are not limited to, IL-1β, IL-6, IL-7, IL-10, IL-12, IL-15, IL-21, or TNFα.

In some embodiments, the additional therapeutic agent comprise, or consists essentially of, or yet further consists of, a receptor agonist. In some instances, the receptor agonist comprise, or consists essentially of, or yet further consists of, a Toll-like receptor (TLR) ligand. In some cases, the TLR ligand comprise, or consists essentially of, or yet further consists of, s TLR1, TLR2, TLR3, TLR4, TLR5, TLR6, TLR7, TLR8, or TLR9. In some cases, the TLR ligand comprise, or consists essentially of, or yet further consists of, s a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some cases, the additional therapeutic agent comprise, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprise, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the VLP formulation described herein is administered in combination with a radiation therapy.

In some instances, the VLP formulation described herein is administered in combination with surgery.

As utilized herein, a pathogen comprise, or consists essentially of, or yet further consists of, a virus, a bacterium, protozoan, helminth, prion, or fungus. In some embodiments, the virus is a DNA virus or an RNA virus. The DNA viruses include single-stranded (ss) DNA viruses, double-stranded (ds) DNA viruses, or DNA viruses that contain both ss and ds DNA regions. The RNA viruses include single-stranded (ss) RNA viruses or double-stranded (ds) RNA viruses. In some cases, the ssRNA viruses are further classified into positive-sense RNA viruses or negative-sense RNA viruses.

Exemplary dsDNA viruses include viruses from the family: Myoviridae, Podoviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxviridae, Sphaerolipoviridae, and Tectiviridae.

Exemplary ssDNA viruses include viruses from the family: Anelloviridae, Bacillariodnaviridae, Bidnaviridae, Circoviridae, Geminiviridae, Inoviridae, Microviridae, Nanoviridae, Parvoviridae, and Spiraviridae.

Exemplary DNA viruses that contain both ss and ds DNA regions include viruses from the group of pleolipoviruses. In some cases, the pleolipoviruses include Haloarcula hispanica pleomorphic virus 1, Halogeometricum pleomorphic virus 1, Halorubrum pleomorphic virus 1, Halorubrum pleomorphic virus 2, Halorubrum pleomorphic virus 3, and Halorubrum pleomorphic virus 6.

Exemplary dsRNA viruses include viruses from the family: Birnaviridae, Chrysoviridae, Cystoviridae, Endornaviridae, Hypoviridae, Megavirnaviridae, Partitiviridae, Picobirnaviridae, Reoviridae, Rotavirus, and Totiviridae.

Exemplary positive-sense ssRNA viruses include viruses from the family: Alphaflexiviridae, Alphatetraviridae, Alvernaviridae, Arteriviridae, Astroviridae, Barnaviridae, Betaflexiviridae, Bromoviridae, Caliciviridae, Carmotetraviridae, Closteroviridae, Coronaviridae, Dicistroviridae, Flaviviridae, Gammaflexiviridae, Iflaviridae, Leviviridae, Luteoviridae, Marnaviridae, Mesoniviridae, Narnaviridae, Nodaviridae, Permutotetraviridae, Picornaviridae, Potyviridae, Roniviridae, Retroviridae, Secoviridae, Togaviridae, Tombusviridae, Tymoviridae, and Virgaviridae.

Exemplary negative-sense ssRNA viruses include viruses from the family: Arenaviridae, Bornaviridae, Bunyaviridae, Filoviridae, Nyamiviridae, Ophioviridae, Orthomyxoviridae, Paramyxoviridae, and Rhabdoviridae.

In some instances, an additional therapeutic agent in the context of a pathogenic infection comprise, or consists essentially of, or yet further consists of, an antibiotics or an antiviral treatments such as, but not limited to, acyclovir, brivudine, docosanol, famciclovir, foscarnet, idoxuridine, penciclovir, trifluridine, valacyclovir, and pritelivir.

In some instances, the pathogen is human immunodeficiency virus (HIV). In some cases, the additional therapeutic agent comprise, or consists essentially of, or yet further consists of, s an HIV antiretroviral therapy. Exemplary HIV antiretroviral therapy includes: nucleoside reverse transcriptase inhibitors (RTIs) such as abacavir, emtricitabine, lamivudine, tenofovir disoproxil fumarate, and zidovudine; non-nucleoside reverse transcriptase inhibitors (NNRTIs) such as efavirenz, etravirine, nevirapine, or rilpivirine; protease inhibitors (Pis) such as atazanavir, darunavir, fosamprenavir, ritonavir, saquinavir, and tipranavir; fusion inhibitors such as enfuvirtide; CCR5 antagonists such as maraviroc; integrase inhibitors such as dolutegravir and raltegravir; post-attachment inhibitors such as ibalizumab; pharmacokinetic enhancers such as cobicistat; and cocktails such as abacavir and lamivudine; abacavir, dolutegravir, and lamivudine; abacavir, lamivudine, and zidovudine; atazanavir and cobicistat; bictegravir, emtricitabine, and tenofovir alafenamide; darunavir and cobicistat; dolutegravir and rilpivirine; efavirenz, emtricitabine, and tenofovir disoproxil fumarate; efavirenz, lamivudine, and tenofovir disoproxil fumarate; efavirenz, lamivudine, and tenofovir disoproxil fumarate; elvitegravir, cobicistat, emtricitabine, and tenofovir alafenamide fumarate; elvitegravir, cobicistat, emtricitabine, and tenofovir disoproxil fumarate; emtricitabine, rilpivirine, and tenofovir alafenamide; emtricitabine, rilpivirine, and tenofovir disoproxil fumarate; emtricitabine and tenofovir alafenamide; emtricitabine and tenofovir disoproxil fumarate; lamivudine and tenofovir disoproxil fumarate; lamivudine and zidovudine; and lopinavir and ritonavir.

In some instances, the pathogen is a hepatitis virus, e.g., hepatitis A, B, C, D, or E. In some cases, an additional therapeutic agent comprise, or consists essentially of, or yet further consists of, an antiviral therapy for hepatitis. Exemplary antiviral therapy for hepatitis include ribavirin; NS3/4A protease inhibitors such as paritaprevir, simeprevir, and grazoprevir; NS5A protease inhibitors such as ledipasvir, ombitasvir, elbasvir, and daclatasvir; NS5B nucleotide/nucleoside and nonnucleoside polymerase inhibitors such as sofosbuvir and dasabuvir; and combinations such as ledipasvir-sofosbuvir, dasabuvir-ombitasvir-paritaprevir-ritonavir; elbasvir-grazoprevir, ombitasvir-paritaprevir-ritonavir, sofosbuvir-velpatasvir, sofosbuvir-velpatasvir-voxilaprevir, and glecaprevir-pibrentasvir; and interferons such as peginterferon alfa-2a, peginterferon alfa-2b, and interferon alfa-2b.

In one aspect, the pathogen is a coronavirus, e.g., COVID-2.

Exemplary autoimmune disease or disorder include, but are not limited to, alopecia areata, autoimmune hemolytic anemia, autoimmune hepatitis, dermatomyositis, type 1 diabetes, juvenile idiopathic arthritis, glomerulonephritis, Graves' disease, Guillain-Barre syndrome, idiopathic thrombocytepenic purpura, myasthenia gravis, multiple sclerosis, pemphigus/pemphigoid, pernicious anemia, polyarteritis nodosa, polymyositis, primary biliary cirrhosis, psoriasis, rheumatoid arthritis, scleroderma, Sjogren's syndrome, systemic lupus erythematosus, thyroiditis, uveitis, vitiligo, or Wegener's granulomatosis.

Exemplary additional therapeutic agents for the treatment of an autoimmune disease or disorder include, but are not limited to, corticosteroids such as prednisone, budesonide, or prednisolone; calcineurin inhibitors such as cyclosporine or tacrolimus; mTOR inhibitors such as sirolimus or everolimus; EVIDH inhibitors such as azathioprine, leflunomide, or mycophenolate; biologies such as abatacept, adalimumab, anakinra, certolizumab, etanercept, golimumab, infliximab, ixekizumab, natalizumab, rituximab, secukinumab, tocilizumab, ustekinumab, or vedolizumab; and monoclonal antibodies such as basiliximab, daclizumab, or muromonab.

Exemplary inflammatory conditions include, but are not limited to, asthma, chronic peptid ulcer, tuberculosis, rheumatoid arthritis, ulcerative colitis, and Crohn's disease.

### Compositions and Formulations

Also provided herein are formulations comprising a VLP as described herein in a gel composition comprising chitosan, alone or in combination with the additional therapeutic agents. In one aspect, the compositions further comprise, or consist essentially of, or yet further consist of, a carrier, such as a pharmaceutically acceptable carrier.

The formulation as described herein comprises a biodegradable polymer, such as low-, medium- or high molecular weight (250-1500 kDa) chitosan mixed with a gel inducer (e.g., disodium glycerophosphate salt) and plant viruses or engineered virus like particles. Chitosan can be incorporated in the formulation as a 0.5-3% aqueous solution in 0.1 M hydrochloric or acetic acid. Disodium glycerophosphate salt is incorporated in the formulation as a 25-75% solution in water for injection. Plant viruses or virus like particles are incorporated as a 5-20 mg mL⁻¹ colloidal solution in sterile buffer saline. The final mixture is a liquid formulation at room temperature, and has a neutral pH (6.8-7.2), gelation time of 2-45 min at 37° C., and viscosity of 0.01-0.90 Pa s when measured using a parallel plate ARG2 rheometer at 25° C.

The following Table 1 shows an example of ingredient contents in the final formulation, but it is important to recognize that modifications thereto falling within the context and spirit of viral particles slow-release concepts are also considered to be within the scope of this disclosure.

TABLE 1

| | | Illustrative |
|---|---|---|
| Ingredient | Content range (%) | content (%) |
| Chitosan | 0.29-1.75 | 1.28 |
| Glycerophosphate | 2.92-8.75 | 5.83 |
| Viral particles | 0.1-1 | 0.45 |
| Hydrochloric/acetic acid | QS. to pH 6.8-7.2 | QS. to pH 6.9 |

The components as provided in this exemplary table are noted in w/v percentage. In the Table, water or a liquid carrier is omitted, but it is understood that the final volume is brought up to 100%. ** QS—bring to.**

In one aspect, the formulation comprises or consists essentially of, virus-like particle (VLP) derived from a plant virus optionally conjugated to a therapeutic peptide, a chitosan polymer and β-glycerophosphate (GP). In another aspect, the formulation comprises the components of Table 1. In an alternative embodiment, the formulation of the gel comprise that identified herein as F1, F2 or F3. In a further aspect, the formulation is F3, identified herein. In another aspect, the chitosan has a molecular weight from about 250 kDa to about 1500 kDa. In a further aspect, the concentration of the VLP in the formulation comprises from about 0.1 mg/ml to about 7 mg/ml at 37° C. In a yet further aspect, the plant virus is from the genus Bromovirus, Comovirus, or Tymovirus. In one aspect, the VLP comprises a capsid protein, optionally a modified capsid protein, further optionally from CCMV, CPMV, PhMV, or a combination thereof. In another aspect, the therapeutic peptide of the formulation comprises an extracellular or transmembrane polypeptide that specifically binds to a receptor expressed on a target cell, optionally an immune cell, optionally wherein the immune cell is an antigen presenting cell, a macrophage, B cell, a dendritic cell, or a nature killer (NK) cell. In one aspect, the macrophage is a tumor-associated macrophage (TAM), that is optionally located within a tumor microenvironment (TME). In one aspect, the formulation further contains an additional therapeutic agent encapsulated within the VLP, optionally a cancer antigen or a peptide that induces an immune response against a pathogen. In a further aspect, the peptide is a cancer antigen or a peptide that induces an immune response against a pathogen. In one aspect, the pathogen is a coronavirus such as SARS-CoV-2. Alternatively, the therapeutic protein comprises a B-cell epitope selected from amino acids 553-570, 625-636 or 809-826 (also identified as 826). In one aspect, the therapeutic protein comprises the B-cell epitope comprising amino acids 809-826 (also identified as 826). In a further aspect of the above formulation, the therapeutic peptide is conjugated to the VLP by a linker, optionally by conjugating the VLP lysine residues to the N-hydroxysuccinimide (NHS)-activated ester of Cy5. In another aspect, the 826 epitope is linked to the VLP through the NHS linkage. In another aspect the formulation comprises a plurality of VLPs. In addition or alternatively, the therapeutic peptides in the plurality are the same or different from each other. In addition or alternatively, the VLPs in the plurality are the same or different from each other.

In a further aspect, the formulation comprises or consists essentially of, virus-like particle (VLP) derived from a plant virus, wherein the plant virus is Cowpea chlorotic mottle virus (CCMV), Cowpea mosaic virus (CPMV), or Physalis mottle virus (PhMV), optionally conjugated to a therapeutic peptide, a chitosan polymer and β-glycerophosphate (GP). In another aspect, the formulation comprises the components of Table 1. In an alternative embodiment, the formulation of the gel comprise that identified herein as F1, F2 or F3. In a further aspect, the formulation comprises F3, as identified herein. In another aspect, the chitosan has a molecular weight from about 250 kDa to about 1500 kDa. In a further aspect, the concentration of the VLP in the formulation comprises from about 0.1 mg/ml to about 7 mg/ml at 37° C. In one aspect, the VLP comprises a capsid protein, optionally a modified capsid protein, further optionally from CCMV, CPMV, PhMV, or a combination thereof. In another aspect, the therapeutic peptide of the formulation comprises an extracellular or transmembrane polypeptide that specifically binds to a receptor expressed on a target cell, optionally an immune cell, optionally wherein the immune cell is an antigen presenting cell, a macrophage, B cell, a dendritic cell, or a nature killer (NK) cell. In one aspect, the macrophage is a tumor-associated macrophage (TAM), that is optionally is located within a tumor microenvironment (TME). In a further aspect, the peptide is a cancer antigen or a peptide that induces an immune response against a pathogen. In one aspect, the pathogen is a coronavirus such as SARS-CoV-2. Alternatively, the therapeutic protein comprises a B-cell epitope selected from amino acids 553-570, 625-636 or 809-826 (also identified herein as 826). In one aspect, the therapeutic protein comprises the B-cell epitope comprising amino acids 809-826 (also identified herein as 826). In a further aspect of the above formulation, the therapeutic peptide is conjugated to the VLP by a linker, optionally by conjugating the VLP lysine residues to the N-hydroxysuccinimide (NHS)-activated ester of Cy5. In another aspect the formulation comprises a plurality of VLPs. In addition or alternatively, the therapeutic peptides in the plurality are the same or different from each other. In addition or alternatively, the VLPs in the plurality are the same or different from each other.

In a further aspect, the formulation comprises or consists essentially of, virus-like particle (VLP) derived from a plant virus, wherein the plant virus is Cowpea chlorotic mottle virus (CCMV), Cowpea mosaic virus (CPMV), or Physalis mottle virus (PhMV), optionally conjugated to a therapeutic peptide, a chitosan polymer and β-glycerophosphate (GP). In another aspect, the formulation comprises the components of F3, identified herein. In another aspect, the chitosan has a molecular weight from about 250 kDa to about 1500 kDa. In a further aspect, the concentration of the VLP in the formulation comprises from about 0.1 mg/ml to about 7 mg/ml at 37° C. In one aspect, the VLP comprises a capsid protein, optionally a modified capsid protein, further optionally from CCMV, CPMV, PhMV, or a combination thereof. In another aspect, the therapeutic peptide of the formulation comprises an extracellular or transmembrane polypeptide that specifically binds to a receptor expressed on a target cell, optionally an immune cell, optionally wherein the immune cell is an antigen presenting cell, a macrophage, B cell, a dendritic cell, or a nature killer (NK) cell. In one aspect, the macrophage is a tumor-associated macrophage (TAM), that is optionally is located within a tumor microenvironment (TME). In a further aspect, the peptide is a cancer antigen or a peptide that induces an immune response against a pathogen. In one aspect, the pathogen is a coronavirus such as SARS-CoV-2. Alternatively, the therapeutic protein comprises a B-cell epitope selected from amino acids 553-570, 625-636 or 809-826 (also identified herein as 826). In one aspect, the therapeutic protein comprises the B-cell epitope comprising amino acids 809-826 (also identified herein as 826) In a further aspect of the above formulation, the therapeutic peptide is conjugated to the VLP by a linker, optionally by conjugating the VLP lysine residues to the N-hydroxysuccinimide (NHS)-activated ester of Cy5, optionally linked to peptide 826. In another aspect the formulation comprises a plurality of VLPs. In addition or alternatively, the therapeutic peptides in the plurality are the same or different from each other. In addition or alternatively, the VLPs in the plurality are the same or different from each other.

In a further aspect, the formulation comprises or consists essentially of, virus-like particle (VLP) derived from a plant virus wherein the plant virus is Cowpea mosaic virus (CPMV), optionally conjugated to a therapeutic peptide, a chitosan polymer and β-glycerophosphate (GP). In another aspect, the formulation comprises the components of Table 1. In a further aspect, the formulation has the components identified herein as F1, F2 or F3. In a further aspect the formulation has the components identified as F3. In another aspect, the chitosan in the formulation has a molecular weight from about 250 kDa to about 1500 kDa. In a further aspect, the concentration of the VLP in the formulation comprises from about 0.1 mg/ml to about 7 mg/ml at 37° C. In another aspect, the therapeutic peptide of the formulation comprises an extracellular or transmembrane polypeptide that specifically binds to a receptor expressed on a target cell, optionally an immune cell, optionally wherein the immune cell is an antigen presenting cell, a macrophage, B cell, a dendritic cell, or a nature killer (NK) cell. In one aspect, the macrophage is a tumor-associated macrophage (TAM), that is optionally is located within a tumor microenvironment (TME). In a further aspect, the peptide is a cancer antigen or a peptide that induces an immune response against a pathogen. In one aspect, the pathogen is a coronavirus such as SARS-CoV-2. Alternatively, the therapeutic protein comprises a B-cell epitope selected from amino acids 553-570, 625-636 or 809-826. In one aspect, the therapeutic protein comprises the B-cell epitope comprising amino acids 809-826. In a further aspect of the above formulation, the therapeutic peptide is conjugated to the VLP by a linker, optionally by conjugating the VLP lysine residues to the N-hydroxysuccinimide (NHS)-activated ester of Cy5, that is optionally linked to peptide 826. In another aspect the formulation comprises a plurality of VLPs. In addition or alternatively, the therapeutic peptides in the plurality are the same or different from each other. In addition or alternatively, the VLPs in the plurality are the same or different from each other.

In a further aspect, the formulation comprises or consists essentially of, virus-like particle (VLP) derived from a plant virus wherein the plant virus is Cowpea mosaic virus (CPMV), optionally conjugated to a therapeutic peptide, a chitosan polymer and β-glycerophosphate (GP). In another aspect, the formulation comprises the components of Table 1. In a further aspect, the formulation has the components identified herein as F1, F2 or F3. In one aspect, the formulation has the components identified as F3 herein. In another aspect, the chitosan in the formulation has a molecular weight from about 250 kDa to about 1500 kDa. In a further aspect, the concentration of the VLP in the formulation comprises from about 0.1 mg/ml to about 7 mg/ml at 37° C. In another aspect, the therapeutic peptide of the formulation comprises a peptide that induces a response to cancer or a peptide that induces an immune response against a pathogen. In one aspect, the pathogen is a coronavirus such as SARS-CoV-2. Alternatively, the therapeutic protein comprises a B-cell epitope selected from amino acids 553-570, 625-636 or 809-826 (also identified herein as peptide 826). In one aspect, the therapeutic protein comprises the B-cell epitope comprising amino acids 809-826 (also identified herein as peptide 826). In a further aspect of the above formulation, the therapeutic peptide is conjugated to the VLP by a linker, optionally by conjugating the VLP lysine residues to the N-hydroxysuccinimide (NHS)-activated ester of Cy5, optionally linked to peptide 826. In another aspect the formulation comprises a plurality of VLPs. In addition or alternatively, the therapeutic peptides in the plurality are the same or different from each other. In addition or alternatively, the VLPs in the plurality are the same or different from each other.

In a further aspect, the formulation comprises or consists essentially of, virus-like particle (VLP) derived from a plant virus, wherein the plant virus is Cowpea mosaic virus (CPMV), a chitosan polymer and β-glycerophosphate (GP), with the proviso that the VLP is not conjugated or joined to a therapeutic peptide. In another aspect, the formulation comprises the components of Table 1. In a further aspect, the formulation has the components identified herein as F1, F2 or F3. In a further aspect, the formulation has the components identified as F3 herein. In another aspect, the chitosan in the formulation has a molecular weight from about 250 kDa to about 1500 kDa. In a further aspect, the concentration of the VLP in the formulation comprises from about 0.1 mg/ml to about 7 mg/ml at 37° C. In another aspect, the VLP comprises a therapeutic peptide that is conjugated to the VLP by a linker, optionally by conjugating the VLP lysine residues to the N-hydroxysuccinimide (NHS)-activated ester of Cy5. In another aspect the formulation comprises a plurality of VLPs. In addition or alternatively, the therapeutic peptides in the plurality are the same or different from each other. In addition or alternatively, the VLPs in the plurality are the same or different from each other.

Also provided are compositions comprising one or more of the formulations as described herein and a carrier, optionally a pharmaceutically acceptable carrier. The formulations can be formulated for in vitro or in vivo use, optionally systemic administration or local administration. For example, the composition is formulated for parenteral administration, optionally for intravenous, subcutaneous, intramuscular, intracerebral, intranasal, intra-arterial, intra-articular, intradermal, intravitreal, intraosseous infusion, intraperitoneal, or intrathecal administration. The composition can further comprise a preservative or stabilizer. In a yet further aspect, the composition or formulation is lyophilized or frozen.

In another aspect, provided herein is a composition comprising, consisting essentially of, or consisting of the combination of formulations comprising a VLP as provided herein, and at least one pharmaceutically acceptable excipient.

In one embodiment, this technology relates to a composition comprising a combination of VLPs or formulations as described herein and a carrier.

In another embodiment, this technology relates to a pharmaceutical composition comprising a combination of VLPs or formulations as described herein and a pharmaceutically acceptable carrier.

In another embodiment, this technology relates to a pharmaceutical composition comprising an effective amount or a therapeutically effective amount of a combination of VLP formulations as described herein and a pharmaceutically acceptable carrier.

Compositions, including pharmaceutical compositions comprising, consisting essentially of, or consisting of the VLP formulation alone or in combination of other therapeutic agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical formulations described herein are administered to a subject by multiple administration routes, including but not limited to, parenteral, oral, buccal, rectal, sublingual, or transdermal administration routes. In some cases, parenteral administration comprise, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL©, dibasic calcium phosphate, dicalcium phosphate dihydrate, tricalcium phosphate, calcium phosphate, anhydrous lactose, spray-dried lactose, pregelatinized starch, compressible sugar, such as Di-PAC© (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL® PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM© HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC© (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Dosage and Dosage Formulations

In some embodiments, the compositions may be administered to a subject suffering from a condition as disclosed herein, such as a human, either alone or as part of a pharmaceutically acceptable formulation, once a week, once a day, twice a day, three times a day, or four times a day, or even more frequently.

Administration of the VLPs formulation alone or in combination with the additional therapeutic agent and compositions containing same can be effected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14, 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

### Diagnostic Methods

In some embodiments, one or more of the methods described herein further comprise, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

The compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

### Uses of the VLPs and Compositions Containing Same

Provided herein is a method of treating a disease or condition or inducing an immune response in a cell or a subject in need thereof, comprising, or consisting essentially of, or yet further consisting of administering to the subject or cell (as appropriate) a VLP formulation or a composition as described herein. In one aspect, the cell or disease or condition is a cancer cell, or a cancer or tumor, e.g. a solid tumor. Non-limiting examples of a solid tumors or cells are bladder cancer, bone cancer, brain cancer, breast cancer, colorectal cancer, colon cancer, esophageal cancer, eye cancer, head and neck cancer, kidney cancer, lung cancer, melanoma, ovarian cancer, pancreatic cancer, prostate cancer, or stomach cancer. In a further aspect, the solid tumor is a colon cancer, pancreatic cancer or melanoma. In a further aspect, the cancer or cell is a hematologic malignancy, such as, for example, a lymphoma or leukemia. Non-limiting examples include a B-cell lymphoma, a T-cell lymphoma, a Hodgkin's lymphoma or a non-Hodgkin's lymphoma. The cancer can be primary or metastatic, e.g., Stage I, Stage II, Stage III or Stage IV. It also can be relapsed or refractory cancer. The cell can be a primary cell obtained from example, a biopsy or an established cell line obtained from example a commercial source such as the American Type Culture Collection (ATCC).

In one aspect, the method or VLP formulation modulates, impedes, or inhibits the growth of a cancer cell or tumor growth. In a yet further aspect, the VLP formulation promotes accumulation of tumor-infiltrating lymphocytes in the TME. In one aspect, the method is practiced in vitro and can serve as an assay to test new combination therapies or dosage regimens. It also can be used as a personalized assay by administering to a subject's cancer or tumor cell in vitro the VLP formulation or composition containing same to the cell. One of skill in the art can use conventional assays, such as reduction in tumor size, tumor burden or a reduction in an appropriate cancer marker to determine if the method is appropriate for the subject in need of such treatment. The methods can be practiced clinically or in laboratory animals as an animal model to test for new combination therapies. One of skill in the art can use conventional assays, such as reduction in tumor size, tumor burden or a reduction in an appropriate cancer marker to determine if the method is appropriate for the subject in need of such treatment or if the treatment has been successful or requires repeating or a change in dosage.

In one aspect, the disease or condition is an infection and the CPMV VLP is conjugated to the B-cell peptide epitope 826 to CPMV (See FIG. 5A) and the formulation is shown in Table 1, or identified herein as F1, F2 or F3. In a further aspect, the formulation is identified herein as F3. An effective amount of the formulation is administered to a subject in need thereof, for example, subcutaneously (S.C.) injection.

In a further aspect, the condition is cancer, optionally colon cancer and the CPMV VLP is formulated according to Table 1 or F3 identified herein. In a further aspect the formulation is F3 and the formulation is by administration by a method described herein, e.g. by implantation in the intraperitoneal space. In addition, an effective amount of the CPMV VLP in a carrier that is not the formulation is administered prior to, concurrently or after the administration of the formulation.

In a further aspect, the VLP formulation modulates secretion of a cytokine from the immune cell, optionally the macrophage, thereby to induce an immunostimulation. In one aspect, the cytokine is a pro-inflammatory cytokine. Non-limiting examples of cytokines are TNFα, IFNγ, IL-1, IL-12, IL-18, or GM-CSF. Methods to measure cytokines are known in the art.

In a further aspect, the method or VLP formulation induces an immune response in the subject in need thereof, e.g., an immune response against a coronavirus, e.g., a COVID-2 infection.

The methods can be practiced clinically or in laboratory animals as an animal model to test for new combination therapies. One of skill in the art can use conventional assays for measuring immune responses.

In the context of cancer care, the subject of these methods can be an animal, a mammal or a human in need of such treatment. When the methods are practiced in vitro, the cell can be an animal cell, a mammalian cell or a human cell. In one aspect an effective amount is administered which can be determined using conventional techniques. When the treatment relates to cancer therapy, the method or treatment can be a first-line, second-line, third-line or fourth-line therapy. In can be an adjuvant therapy and combined with other therapies as determined by the treating veterinarian or physician. Any appropriate means of administration can be used, also as determined by the treating veterinarian or physician. The treatments can be combined with diagnostic assessment before or after therapy. Thus, the therapy can be personalized to the subject in need of such treatment.

Provided herein is a method of treating an inflammatory condition in a subject in need thereof, comprising, or consisting essentially of, or yet further consisting of administering to the subject an VLP or a composition as described herein. Further provided is a method of treating an autoimmune disease in a subject in need thereof, comprising, or consisting essentially of, or yet further consisting of administering to the subject a VLP formulation or a composition as described herein. The methods can be practiced clinically or in laboratory animals as an animal model to test for new combination therapies. Methods to measure inflammatory responses are known in the art.

Yet further provided is a method of treating an allergy in a subject in need thereof, comprising, or consisting essentially of, or yet further consisting of administering to the subject a VLP formulation or a composition as described herein. The disease or condition is an inflammatory condition. In one aspect, the allergy, comprises asthma, allergic asthma or allergic rhinosinusitis. The methods can be practiced clinically or in laboratory animals as an animal model to test for new combination therapies. Methods to measure allergic responses are known in the art.

Still further provided is a method of treating a pathogenic condition in a subject in need thereof, comprising, or consisting essentially of, or yet further consisting of administering to the subject a VLP formulation or a composition as described herein. In one aspect, the pathogen is a virus, e.g., a coronavirus, a human immunodeficiency virus (HIV) or a Hepatitis virus, optionally a Hepatitis B virus or a Hepatitis C virus. In another aspect, the pathogen is a bacterium, protozoan, helminth, prion, or fungus. Non-limiting examples of such include Vibrio parahaemolyticus or rock bream iridovirus, Edwardsiella tarda, or Vibrio vulnificus.

The methods can be practiced clinically or in laboratory animals as an animal model to test for new combination therapies. Methods to measure pathogenic infection are known in the art.

Further provided is a method of modulating phagocytosis in a target cell, comprising, or consisting essentially of, or yet further consisting of contacting the target cell or a plurality of target cells comprising, or consisting essentially of, or yet further consisting of a macrophage with an VLP formulation or composition containing same for a first time sufficient to activate phagocytic activity of the macrophage and contacting the activated macrophage with the target cell for a second time sufficient to induce phagocytosis of the target cell. In one aspect, the macrophage has a M1 phenotype. In another aspect, the target cell or the plurality of cells are located in a tumor microenvironment (TME). In a further aspect, the cell or the plurality of cells comprise, or consists essentially of, or yet further consists of, antigen-presenting cells (APCs), non-limiting examples of such include dendritic cells, B cells, or a combination thereof. In a further aspect, the target cell or population comprise a cancer cell.

In one aspect, the target cell or population comprising the target cell comprises a cancer cell, or a cancer or tumor, e.g. a solid tumor. Non-limiting examples of a solid tumors or cells are bladder cancer, bone cancer, brain cancer, breast cancer, colorectal cancer, colon cancer, esophageal cancer, eye cancer, head and neck cancer, kidney cancer, lung cancer, melanoma, ovarian cancer, pancreatic cancer, prostate cancer, or stomach cancer. In a further aspect, the solid tumor is a colon cancer, pancreatic cancer or melanoma. In a further aspect, the cancer or cell is a hematologic malignancy, such as, for example, a lymphoma or leukemia. Non-limiting examples include a B-cell lymphoma, a T-cell lymphoma, a Hodgkin's lymphoma or a non-Hodgkin's lymphoma. The cancer can be primary or metastatic, e.g., Stage I, Stage II, Stage III or Stage IV. It also can be relapsed or refractory cancer. The cell can be a primary cell obtained from example, a biopsy or an established cell line obtained from example a commercial source such as the American Type Culture Collection (ATCC).

In one aspect, the method inhibits the growth of a cancer cell or tumor growth. In one aspect, the method is practiced in vitro and can serve as an assay to test new combination therapies or dosage regimens. It also can be used as a personalized assay by administering to a subject's cancer or tumor cell in vitro the VLP formulation or composition containing same to the cell. One of skill in the art can use conventional assays, such as reduction in tumor size, tumor burden or a reduction in an appropriate cancer marker to determine if the method is appropriate for the subject in need of such treatment or if the treatment has been successful or requires repeating or a change in dosage. The methods can be practiced clinically or in laboratory animals as an animal model to test for new combination therapies. One of skill in the art can use conventional assays, such as reduction in tumor size, tumor burden or a reduction in an appropriate cancer marker to determine if the method is appropriate for the subject in need of such treatment.

The subject of these methods can be an animal, a mammal or a human in need of such treatment. When the methods are practiced in vitro, the cell can be an animal cell, a mammalian cell or a human cell. In one aspect an effective amount is administered which can be determined using conventional techniques. When the treatment relates to cancer therapy, the method or treatment can be a first-line, second-line, third-line or fourth-line therapy. In can be an adjuvant therapy and combined with other therapies as determined by the treating veterinarian or physician. Any appropriate means of administration can be used, also as determined by the treating veterinarian or physician. The treatments can be combined with diagnostic assessment before or after therapy. Thus, the therapy can be personalized to the subject in need of such treatment.

In another aspect, the target cell or plurality of target cells comprise a cell infected by a pathogen. In one aspect, the pathogen is a virus, e.g., a coronavirus, a human immunodeficiency virus (HIV) or a Hepatitis virus, optionally a Hepatitis B virus or a Hepatitis C virus. In another aspect, the pathogen is a bacterium, protozoan, helminth, prion, or fungus. Non-limiting examples of such include Vibrio parahaemolyticus or rock bream iridovirus, Edwardsiella tarda, or Vibrio vulnificus. The methods can be practiced clinically or in laboratory animals as an animal model to test for new combination therapies. Methods to measure pathogenic infection are known in the art.

Also provided is a method of modulating M1 macrophage polarization, comprising, or consisting essentially of, or yet further consisting of contacting a plurality of antigen presenting cells (APCs) comprising, or consisting essentially of, or yet further consisting of at least one macrophage with an VLP formulation or composition as described herein for a time sufficient to induce secretion of a plurality of cytokines by the plurality of APCs, whereby the secretion of the plurality of cytokines modulate M1 activation of the macrophage. In one aspect, the APCs are located within a tumor microenvironment. In another aspect, the plurality of cytokines comprise, or consists essentially of, or yet further consists of IFNγ, TNFα, or a combination thereof. In a further aspect, the VLP formulation or composition decreases M2 activation of the macrophage. In a yet further aspect, the APCs further comprise, or consists essentially of, or yet further consists of dendritic cells, B cells, or a combination thereof.

In one aspect, the method is practiced in vitro. In another aspect, the method is an in vivo method. In a further aspect, the method is an ex vivo method.

In one aspect, the method is practiced in vitro and can serve as an assay to test new combination therapies or dosage regimens. The methods can be practiced clinically or in laboratory animals as an animal model to test for new combination therapies. One of skill in the art can use conventional assays, to determine efficacy. The subject of these methods can be an animal, a mammal or a human in need of such treatment.

When the methods are practiced in vitro, the cell can be an animal cell, a mammalian cell or a human cell. In one aspect an effective amount is administered which can be determined using conventional techniques. When the treatment relates to cancer therapy, the method or treatment can be a first-line, second-line, third-line or fourth-line therapy. In can be an adjuvant therapy and combined with other therapies as determined by the treating veterinarian or physician. Any appropriate means of administration can be used, also as determined by the treating veterinarian or physician. The treatments can be combined with diagnostic assessment before or after therapy. Thus, the therapy can be personalized to the subject in need of such treatment.

In some embodiments as described herein, the VLP or compositions described herein are administered for clinical or therapeutic applications.

In some embodiments, the VLP formulation or composition as described herein are administered to a subject by multiple administration routes, including but not limited to, parenteral, oral, buccal, rectal, sublingual, or transdermal administration routes. In some cases, parenteral administration comprise, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the VLP formulation or composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits

As used herein, a kit or article of manufacture described herein include a carrier, package, or container that is compartmentalized to receive one or more containers such as vials, tubes, and the like, each of the container(s) comprising, or consisting essentially of, or yet further consisting of, one of the separate elements to be used in a method described herein. Suitable containers include, for example, bottles, vials, syringes, and test tubes. In one embodiment, the containers are formed from a variety of materials such as glass or plastic. The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Experimental

These examples are provided for illustrative purposes only and not to limit the scope of the claims provided herein.

### Experiment No. 1

The RBD is the binding site for most neutralizing antibodies against SARS-CoV-2.³⁸ Applicant previously demonstrated that three B-cell epitopes (peptide sequences 553-570, 625-636 and 809-826), which are common to many SARS-CoV-2 variants, are suitable for the development of effective pan-specific vaccines against SARS-CoV-2.³⁹ To enhance the immune response, these peptide epitopes were attached to cowpea mosaic virus (CPMV) or virus-like particles (VLPs) derived from bacteriophage Qβ, which function as a combined adjuvant and epitope nanocarrier, promoting trafficking across draining lymph nodes and interactions with antigen presenting cells.^{40,41} CPMV has bipartite RNA genome encapsulated in a 30-nm icosahedral capsid consisting of 60 asymmetrical copies of small (24 kDa) and large (41 kDa) coat protein (CP) subunits.⁴² Both the capsid and RNA are immunostimulatory, therefore rendering CPMV a potent adjuvant. For example, the strong immunogenicity of native CPMV^{44,45} makes it an effective in situ vaccine against various tumors in mouse models^{41,46,47} and canine patients.⁴⁸ It also serves as a delivery platform and multiple copies of the SARS-CoV-2 peptide epitopes can be displayed via chemical bioconjugation.⁴³ When tested as soluble prime-boost formulations, microneedle patches or slow-release poly(lactic-co-glycolic acid) (PLGA) implants, the CPMV- and Qβ-based COVID-19 vaccine candidates formulations elicited neutralizing antibodies against SARS-CoV-2, and the soluble prime-boost vaccine (CPMV conjugated to epitope sequence 809-826) elicited a neutralization titer comparable to Moderna's mRNA-1273 vaccine.³⁹ The Qβ formulation maintained efficacy when formulated as a PLGA implant, but in a previous study with a similar approach against SARS-CoV the efficacy of CPMV-based vaccines declined significantly in this format when administered as a single dose.⁴³ This reflected the lower immunostimulatory response caused by the loss of CPMV RNA during freeze-drying, as required for implant formulation.⁴⁹ The efficacy of a CPMV-based vaccine displaying the 809-826 epitope sequence (826-CPMV) could perhaps be improved by investigating alternative single-dose formulations, such as those based on the natural biopolymer chitosan.

Chitosan is a polysaccharide produced by the deacetylation of chitin.⁵⁰ It is generally regarded as safe (GRAS) as an excipient, and is therefore considered to be biocompatible, non-immunogenic and biodegradable.^{51,52} It is already approved for products such as BST-CarGel for the regeneration of cartilage.⁵³ Many studies have reported excellent immune-enhancing capability of chitosan as a vaccine adjuvant for nasal,⁵⁴ parenteral,⁵⁵ and subcutaneous administrations.⁵⁶ Chitosan-based hydrogels are produced by mixing chitosan with β-glycerophosphate (GP) to yield liquid formulations that are fluid at room temperature but form a gel at body temperature. This thermo-responsive behavior is driven by the interactions between GP and the polar backbone of chitosan, which prevents polymer precipitation, balances the pH and triggers gelation when heated.⁵⁷⁻⁵⁹ Such thermo-responsive hydrogels are advantageous because they are simple to prepare and inject.^{60,61} Chitosan/GP hydrogels have been extensively used for drug delivery,^{62,63} tissue regeneration/repair,^{64,65} and the slow release of nanoparticles.^{66,67}

Applicant discloses the development of an in situ forming chitosan/GP hydrogel loaded with 826-CPMV as a single-dose vaccine against COVID-19. Applicant initially prepared chitosan/GP hydrogels containing native CPMV particles for formulation design and optimization before testing CPMV labeled with the fluorophore sulfo-cyanine 5 (Cy5) as a cargo model for the characterization of in vitro/in vivo release profiles by fluorescence analysis. 826-CPMV particles formulated as chitosan/GP hydrogels were then prepared and used to immunize BALB/c mice subcutaneously. The antibody response was monitored for 20 weeks, comparing the hydrogel to soluble formulations in terms of antibody titers and subtypes.

Preparation of native CPMV. CMPV was propagated in and extracted from the leaves of black-eyed pea plants (Vigna unguiculata) as previously described.^{68,69} Frozen leaf tissue (100 g) was homogenized in 300 mL 0.1 M potassium phosphate (KP) buffer (pH 7.0), then filtered and centrifuged (18,500×g, 20 min, 4° C.) to remove plant debris. The supernatant was extracted with 1:1 chloroform:1-butanol and the aqueous phase was mixed with 0.2 M NaCl and 8% PEG 8000 for CPMV precipitation. The mixture was centrifuged (30,000×g, 15 min, 4° C.) and the pellet was resuspended in 0.01 M KP buffer. After a further round of centrifugation (13,500×g, 15 min, 4° C.) to remove aggregates, the supernatant was purified on a 10-40% sucrose gradient. The bright bands were isolated and purified by ultracentrifugation (42,000 rpm, 2.5 h, 4° C.) using an Optima L-90K centrifuge with rotor type 50.2 Ti (Beckman Coulter, Brea, CA, USA). CPMV particles were dispersed in 0.1 M KP buffer and the CP concentration was determined in a NanoDrop 2000 UV/visible spectrophotometer (Thermo Fisher Scientific, Waltham, MA, USA) at 260 nm using a molar extinction coefficient (6260 nm) of 8.1 mg⁻¹ mL cm⁻¹.

Conjugation of CPMV to sulfo-Cy5. Cy5-CPMV particles were prepared by conjugating CPMV lysine residues to the N-hydroxysuccinimide (NHS)-activated ester of Cy5 (Lumiprobe, Hunt Valley, MD, USA). Covalent attachment was achieved by reacting 25 μL 50 mg mL⁻¹ NHS-Cy5 (5 equivalents per CP) with 10 mg CPMV in 0.01 M KP buffer on an orbital shaker for 2 h at room temperature. The Cy5-CPMV conjugate was continuously purified using a 100-kDa molecular weight cutoff (MWCO) centrifugal filter (500×g, 5 min, room temperature) until a clear filtrate was obtained. The concentration of Cy5-CPMV particles was determined by UV-vis spectrophotometry as above, and the Cy5 absorption at 647 nm (ε_{647 nm}=271 000 L mol⁻¹ cm⁻¹) was used to estimate the dye loading per particle.

Conjugation of CPMV to epitope 826. CPMV particles were labeled with the bifunctional PEGylated cross-linker SM(PEG)₄ (Thermo Fisher Scientific) using a reactive NHS-activated ester that targets lysine residues. The reaction was done by mixing 2000-fold molar excess of SM(PEG)₄ with 2 mg CPMV particles in 0.01 M KP buffer for 2.5 h at room temperature. The PEGylated intermediate was purified using a 100-kDa MWCO centrifugal filter (16,000×g, 5 min, 4° C.). The maleimide handles of the PEGylated intermediate were then reacted with the cysteine residue of epitope 826 (GenScript Biotech, Piscataway, NJ, USA) by mixing 2 mg PEGylated CPMV with 0.2 mL 20% Pluronic F-127 (MilliporeSigma, Burlington, MA, USA) in DMSO⁷⁰ and then adding 0.12 mL 20 mg mL⁻¹ epitope 826 in DMSO and stirring overnight. The 826-CPMV conjugate was purified by centrifugation on a 0.1-mL 40% sucrose cushion (50,000 rpm, 1 h, 4° C.) and dialysis against 0.01 M KP buffer for 24 h at room temperature. The 826-CPMV particles were concentrated using a 100-kDa MWCO centrifugal filter (8000×g, 5 min, 4° C.) and quantified by UV-vis spectrophotometry as above. They were also visualized by transmission electron microscopy (TEM) on a Tecnai F30 instrument (FEI Company, Hillsboro, OR, USA) after staining with 2% uranyl acetate.

### Characterization of CPMV Nanoparticles

Size exclusion chromatography. 200 μg CPMV particles were loaded onto a Superose6 column in the ÄKTA Explorer chromatography system (GE Healthcare, Chicago, IL, USA) and eluted them in 0.1 M KP buffer (pH 7.0) at a flow rate of 0.5 mL min⁻¹. The capsid protein, viral RNA and conjugated Cy5 dye were detected at 260, 280, and 647 nm, respectively.

Dynamic light scattering (DLS). The hydrodynamic diameter, polydispersity index (PDI) and zeta potential of the particles were determined using a Zetasizer Nano ZSP Zen5600 instrument (Malvern Panalytical, Malvern, UK). Triplicate measurements were acquired over 3-5 min at room temperature with a scattering angle of 90°.

Native gel electrophoresis. Particles (10-20 g) suspended in Tris/Borate/EDTA (TBE) buffer (45 mM Tris, 45 mM boric acid, 1.25 mM EDTA in Milli-Q water) were loaded onto 1.2% agarose gels and fractionated for 30 min at 120 V and 400 mA. Gels were documented on an AlphaImager (Protein Simple, San Jose, CA, USA) under UV, red and white light before and after staining with Coomassie Brilliant Blue (CBB).

Sodium dodecylsulfate polyacrylamide gel electrophoresis (SDS-PAGE). Protein samples (10 g) were analyzed side by side with SeeBlue Plus2 pre-stained protein standards (Thermo Fisher Scientific) on 4-12% or 12% NuPAGE polyacrylamide gels using 1×MOPS elution buffer (Invitrogen, Thermo Fisher Scientific) at 200 V and 120 mA for 40 min. Gel images were documented on the AlphaImager system under red and white light before and after CBB staining.

### Hydrogel Formulation and Characterization

Preparation of chitosan/GP formulations. Liquid formulations were prepared by mixing the chitosan and GP solutions and vortexing the mixture with the CPMV, Cy5-CPMV or 826-CPMV particles. The chitosan solution was prepared by dispersing 4 g of chitosan powder (Chem-Impex International, Wood Dale, IL, USA) in 180 mL 0.1 M HCl for 2 h, followed by autoclaving for 20 min at 121° C. and homogenization by stirring overnight at room temperature).⁷¹ Chitosan solutions of low molecular weight (LMW, 250 kDa), medium molecular weight (MMW, 1250 kDa) and high molecular weight (HMW, 1500 kDa) were prepared. The GP solution was prepared by dissolving 5.60 g β-glycerophosphoric acid disodium salt (MilliporeSigma) in 10 mL deionized water and passing the solution through a 0.22-μm filter. The chitosan and GP solutions were mixed at a 5:1 (v/v) ratio,⁶⁴ and different amounts of CPMV in PBS were dispersed by vortexing to yield 0 (blank), 2.25 (0.225%) and 4.5 mg mL⁻¹ (0.450%) of CPMV nanoparticles in the final formulations (Table 2). Minitab v13 (Minitab, Coventry, UK) was used for the factorial design of nine different formulations for evaluation against gelation time. CPMV 0.45% was duly selected and the Cy5-CPMV formulations were prepared as follows: chitosan/GP solutions were vortexed with 15 mg mL⁻¹ Cy5-CPMV at a 7:3 (v/v) ratio yielding 0.45% formulations denoted F1, F2 and F3 representing the LMW, MMW and HMW chitosan, respectively. Formulation F3 based on HMW chitosan achieved the shortest gelation time and prolonged release profiles, and was therefore used to encapsulate 826-CPMV as described for Cy5-CPMV. Blank hydrogels were prepared under the same conditions using PBS lacking CPMV particle.

TABLE 2
Formulation parameters for the design
of CPMV/chitosan/GP hydrogels.

| | Chitosan molecular | Final CPMV concentration, |
|---|---|---|
| Level | weight (MW) | mg mL⁻¹ (%) |
| 1 | Low MW (250 kDa) | 0 (0%) |
| 2 | Medium MW (1250 kDa) | 2.25 (0.225%) |
| 3 | High MW (1500 kDa) | 4.5 (0.45%) |

Viscosity measurements. Viscosity was measured using a parallel plate ARG2 rheometer (TA Instruments, New Castle, DE, USA). 200 L of each sample were pipetted into the center of the parallel plate geometry, which was set at 25° C. with a gap height of 500 m (ensuring the liquid covered the entire gap between the plates).

Determination of gelation time using the tube inversion method. 1 mL of each sample was added (in a 1.5-mL Eppendorf tube) at 37° C. and inverted the tube every 60 s. The gelation time point was recorded when the formulation no longer flowed in the inverted tube after 30 s.⁶⁶

Hydrogel swelling and degradation in vitro. Applicant incubated 0.5 mL of each hydrogel sample containing Cy5-CPMV (in a 1.5-mL Eppendorf tube) at 37° C. for 45 min to ensure complete gelation. The initial height of the gel was measured before carefully adding 1 mL PBS and agitating the tubes at 200 rpm. At predefined time intervals, the liquid phase was removed and set aside for Cy5-CPMV characterization. The same amount of fresh PBS was added and the height of gel was recorded to calculate the swelling ratio (the height at any time divided by the initial height×100).⁶⁶ Following this longitudinal incubation in PBS, exhausted gels (and fresh gels) were freeze-dried and imaged by scanning electron microscopy (SEM) using a Quanta 600 ESEM (FEI Company) operating at 10 kV.

Characterization of Cy5-CPMV released from hydrogels in vitro. The liquid phase set aside from the previous step was compared to a defined amount of Cy5-CPMV in PBS as a control. Fluorescence measurements were recorded on a microplate reader (Tecan, Mannedorf, Switzerland) to quantify Cy5 (λEx=600 nm, λEm=665 nm) and estimate Cy5-CPMV release profiles.⁶⁷ The particles were separated by SDS-PAGE to confirm molecular stability of the Cy5-CPMV CPs conjugates. The intactness of the particles was confirmed by native gel electrophoresis and TEM as described above.

### Animal Experiments

Ethical statements. Animal procedures were carried out according to the guidelines of the Institutional Animal Care and Use Committee (IACUC) of the University of California San Diego (UCSD), following the protocols approved by the Animal Ethics committee of UCSD. For all animal experiments, Applicant used healthy BALB/c female mice (7-8 weeks old) purchased from the Jackson Laboratory (Bar Harbor, ME, USA) and hosted at the UCSD Moores Cancer Center with unlimited food and water.

Characterization of Cy5-CPMV released from hydrogels in vivo. Hydrogel formulations F1-F3 (100 μL, containing 450 g Cy5-CPMV) or soluble Cy5-CPMV (450 g in 100 μL PBS) were administered as single subcutaneous injections behind the neck of shaved mice on day 0 (five mice per group). Animals were maintained on an alfalfa-free diet 1 week before the experiment and throughout the study to prevent tissue autofluorescence. The injection site was imaged at different time points under a Xenogen IVIS 200 Optical Imaging System (Caliper Life Sciences, Hopkinton, MA, USA). IVIS software was used to determine the fluorescence intensity within a region of interest (ROI) and thus evaluate the persistence of fluorescence as a marker of slow release. The F3 formulation (200 g single subcutaneous injection) was then selected for comparison to 2×100 μg doses of soluble Cy5-CPMV.

Immunization procedure. BALB/c female mice (four mice per group) were assigned to one of the following treatment groups, with all treatments involving subcutaneous injections behind the neck: (i) group 100=prime-boost (week 0 and week 2) injections of 100 g soluble 826-CPMV in 150 μL PBS; (ii) group 200=single injection of 200 g soluble 826-CPMV in 150 μL PBS; (iii) group F3=single injection of the F3 formulation containing 200 g 826-CPMV; and (iv) group blank F3=single injection of the F3 formulation without 826-CPMV. Blood samples were collected by retro-orbital bleeding before injection (week 0) and on weeks 2, 4, 8, 12, 16 and 20. Blood samples were centrifuged (2000×g, 10 min, 4° C.) and the plasma was kept at −80° C. for antibody screening.

Enzyme-linked immunosorbent assay (ELISA). Anti-826 antibodies were detected by ELISA as previously reported.³⁹ Pierce maleimide-activated 96-well plates (Thermo Fisher Scientific) were rinsed three times with 200 μL per well PBS containing 0.05% (v/v) Tween-20 (PBST), and the same washing procedure was used between all subsequent steps. The washed plates were coated with peptide epitope 826 (20 g mL⁻¹, 100 μL per well) in binding buffer (0.1 M sodium phosphate, 0.15 M sodium chloride, 0.01 M EDTA, pH 7.2) overnight at 4° C. After discarding the coating solution and washing the plates, each well was blocked with 100 μL 10 μg mL⁻¹ cysteine in binding buffer, and the plates were incubated at room temperature for 1 h. Following the blocking step, plasma from immunized animals was added in PBS (100 μL per well) using dilution factors of 200, 400, 800, 1600, 3200, 6400, 12,800, 25,600, 51,200, 102,400 and 204,800. After incubating for 1 h at room temperature and washing, Applicant added the horseradish peroxidase (HRP)-conjugated goat anti-mouse IgG Fc-specific secondary antibody (Invitrogen, diluted 1:5000) in PBST and incubated the plates again for 1 h at room temperature. Following another wash, Applicant added 100 μL per well of the 1-Step Ultra TMB-ELISA substrate (Thermo Fisher Scientific) and allowed the plates to develop for 5 min at room temperature before stopping the reaction with 100 μL per well of 2 N H₂SO₄ and reading the optical density at 450 nm on a Tecan microplate reader.

Antibody isotyping. The ELISA protocol for anti-826 antibody screening was slightly modified for the isotyping experiment. Instead of serial dilutions, samples from weeks 4 and 12 were diluted 1:1000 in binding buffer. As secondary antibodies, Applicant used HRP-conjugated goat anti-mouse IgG1 (Invitrogen PA174421, 1:5000), IgG2a (Invitrogen A-10685, 1:1000), IgG2b (Abcam, Cambridge, UK, ab97250, 1:5000), IgG2c (Abcam ab9168, 1:5000), IgG3 (Abcam ab98708, 1:5000), IgE (Invitrogen PA184764, 1:1000), and IgM (Abcam ab97230, 1:5000). The IgG1/IgG2a ratio was calculated, with values <1 considered indicative of a Th1 response and values >1 considered indicative of a Th2 response.

### Statistical Analysis

Graphical data were processed and analyzed using GraphPad Prism v9.0.2 (GraphPad Software, San Diego, CA, USA), unless otherwise indicated. Depending on the datasets, data were statistically compared by one-way analysis of variance (ANOVA) followed by Tukey's multiple comparison test or two-way ANOVA using pairwise multiple comparison followed by a post-test Holm-Šidák correction. Asterisks in figures indicate significant differences between groups (*p<0.05; **p<0.01; ***p<0.001; ****p<0.0001).

### Results and Discussion

### Preparation and Labeling of CPMV Particles

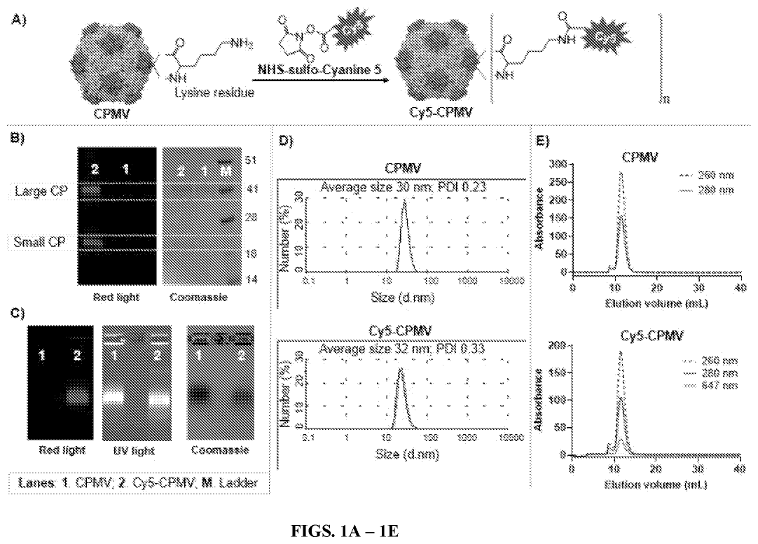

CPMV was purified from infected black-eyed pea plants yielding 0.55 mg per gram of leaf tissue. The 260/280 nm absorbance ratio was 1.75, well within the 1.7-1.8 range anticipated for pure particles.⁶⁹ Surface-exposed lysine side chains were conjugated to Cy5 using NHS chemistry (FIG. 1A). Five equivalents of NHS-sulfo-Cy5 per CP achieved a loading efficiency of 19 Cy5 molecules per particle, which is acceptable for fluorescence imaging.¹² SDS-PAGE and native agarose gel electrophoresis confirmed the attachment of Cy5 (FIG. 1B, FIG. 1C). Illumination of the polyacrylamide gels with red light revealed fluorescent bands matching the small and large CP bands on gels stained with CBB, indicating that Cy5 was covalently linked to both polypeptides. Illumination of the native agarose gels under red light showed a fluorescent band matching the UV band (RNA signal) and the protein band on gels stained with CBB (intact particles), thus confirming that the particles were intact following bioconjugation. This was consistent with size analysis by DLS, which showed the presence of nanometer-scale particles in the CPMV and Cy5-CPMV samples (FIG. 1D). Particle integrity was verified by the single elution peak during size exclusion chromatography: proteins were detected at 260 nm, RNA at 280 nm and Cy5-CPMV at 647 nm (FIG. 1E). The latter also confirmed the absence of aggregates, broken particles, free proteins or free dye molecules.

Preparation and Characterization of Hydrogels Loaded with CPMV/Cy5-CPM

Gel formation. Chitosan is soluble in acids due to the electrostatic repulsion between its positively charged, amine-protonated chains. The addition of GP neutralizes the solution (pH=6.5-7.3) without inducing immediate precipitation or aggregation because GP deprotonates some of chitosan's positively charged amine groups (—NH₃+), allowing electrostatic attraction between the GP phosphate backbone and chitosan's remaining —NH₃+ groups, in turn exposing the glycerol moiety of GP to neighboring chitosan chains and enhancing their solubility when the temperature is below ˜23° C.^{73,57} Higher temperatures trigger the transfer of protons from chitosan's —NH₃+ groups to the GP phosphate backbone, reducing the charge density of chitosan and favoring hydrophobic inter-chain interactions and hydrogen bonding between chitosan chains, resulting in the formation of a gel.^{50,59,73,7}

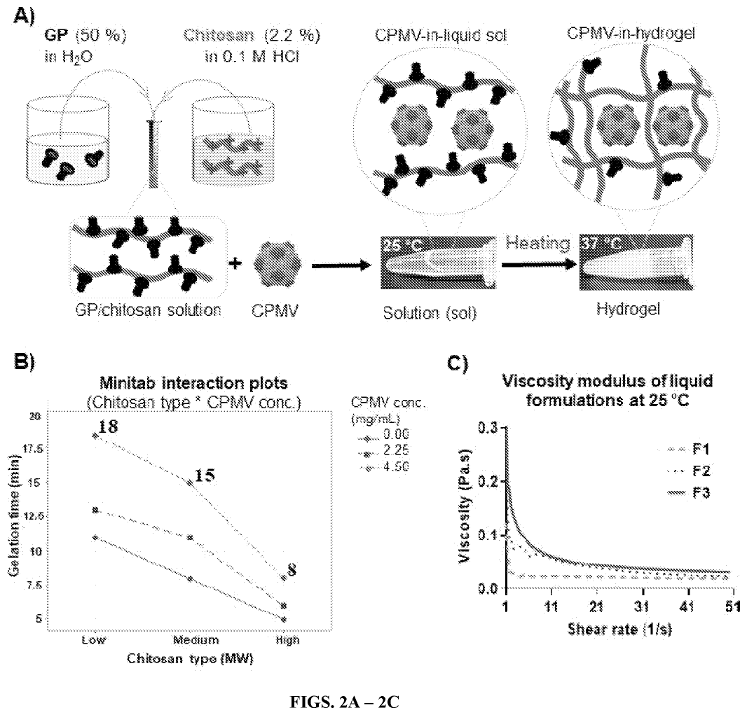

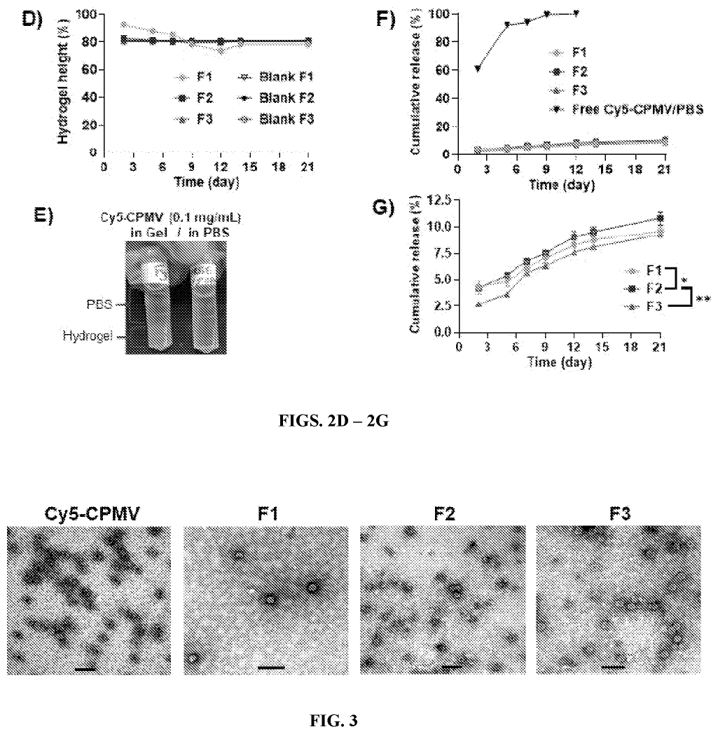

Applicant investigated the gelling behavior of chitosan/GP mixtures featuring three different molecular weights of chitosan (LMW=250 kDa, MMW=1250 kDa and HMW=1500 kDa) and various concentrations of CPMV (0-4.5 mg mL⁻¹) at 37° C. The gelation time was assessed by the flow and turbidity of each mixture following tube inversion (FIG. 2A). The gelation time decreased with increasing chitosan molecular weight, but the concentration of CPMV was also relevant (FIG. 2B). This is consistent with previous studies demonstrating that solution-to-gel transition is influenced by many formulation parameters, including chitosan molecular weight and cargo loading.⁷⁵ Blank formulations gelled much faster than those containing CPMV, supporting previous observations that nanoparticles occupy the space between chitosan chains and slow gelation.⁶⁷ The shortest gelling time was observed for the formulations containing HMW chitosan (5-8 min). Applicant selected the formulations with the highest load of CPMV (4.5 mg mL⁻¹) for further characterization because this allows the maximum dosage with the smallest volume of excipient. The formulations containing 4.5 mg mL⁻¹ CPMV dispersed in LMW, MMW and HMW chitosan were named F1, F2 and F3, respectively. The liquid formulation F3 was the most viscous (0.482 Pa·s), 2.4-fold more viscous than F2 (0.202 Pa·s) and 4.8-fold more than F1 (0.099 Pa·s).

The viscosity modulus of F1 (and to some degree F.2) decreased abruptly as the shear rate increased, whereas the viscosity modulus of F3 declined gradually (FIG. 2C). This indicates much better shear-thinning and self-healing behavior,⁶³ reflecting the presence of stronger inter-chain interactions as would be anticipated from the short gelation time.

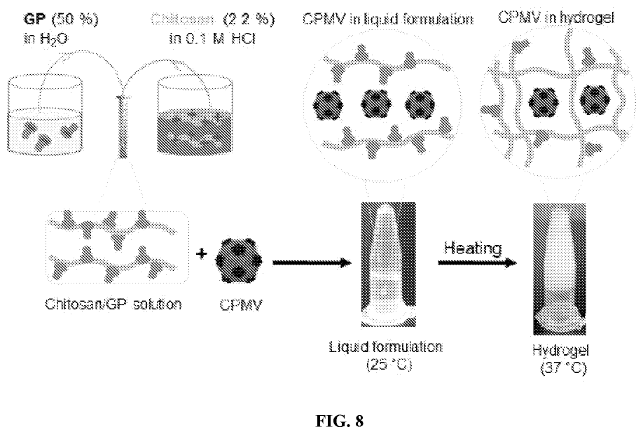

Gel swelling, degradation, and in vitro release profiles. Next, Applicant assessed gel swelling and degradation, as well as the Cy5-CPMV release profile over 21 days in PBS at 37° C. Although hydrogel F1 initially showed some fluctuations (FIG. 2D), all formulations ultimately showed no significant change in gel height (FIG. 2E). The apparent volume of the gel therefore remained constant regardless of the composition (loaded with Cy5-CPMV particles or blank). This agrees with one earlier report⁶⁶ but in another case the authors observed significant height fluctuations.⁷⁶ The constant apparent volume of our gel suggests that the rates of gel swelling and degradation are comparable, which implies a robustness that may interfere with cargo release. However, SEM revealed that the microstructure of fresh (non-incubated) hydrogels comprised a bulky but porous matrix, which would encourage cargo release even without degradation (FIG. 8). SEM images of exhausted gels (after incubation in PBS) included abundant salt crystals, which made it difficult to determine the matrix structure (data not shown). Despite these results, the slow-release capability of the hydrogels was confirmed directly by measuring the quantity of Cy5-CPMV particles in the liquid phase (FIG. 2E). The gels remained stable throughout the 21 days of testing, but Applicant observed the gradual release of Cy5-CPMV nanoparticles from all formulations, suggesting the particles can diffuse through the pores identified above (FIG. 2F, FIG. 2G). The slowest release profile was observed for F3, consistent with its rapid gelation and high viscosity, followed by F1 and then F2. This suggests the release profile is not directly related to the molecular weight of chitosan. Applicant found that a free suspension of Cy5-CPMV released 100% of the particles after incubation in PBS for 10 days, which was anticipated because the particles can move freely due to Brownian motion. In contrast, only 10-12% of the particles were released from the hydrogels after 21 days, reflecting a combination of physical obstruction and chemical interactions within the gel matrix.^{77,78}

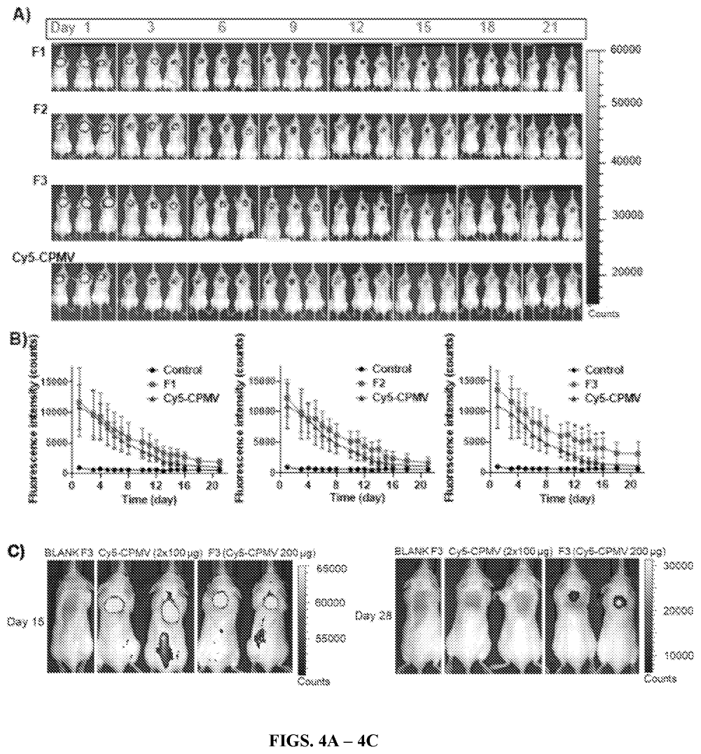

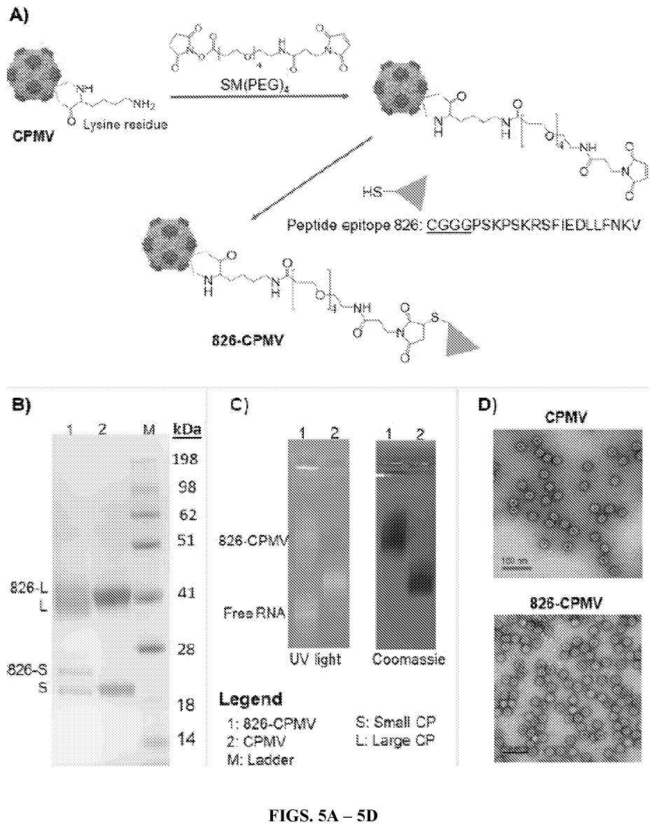

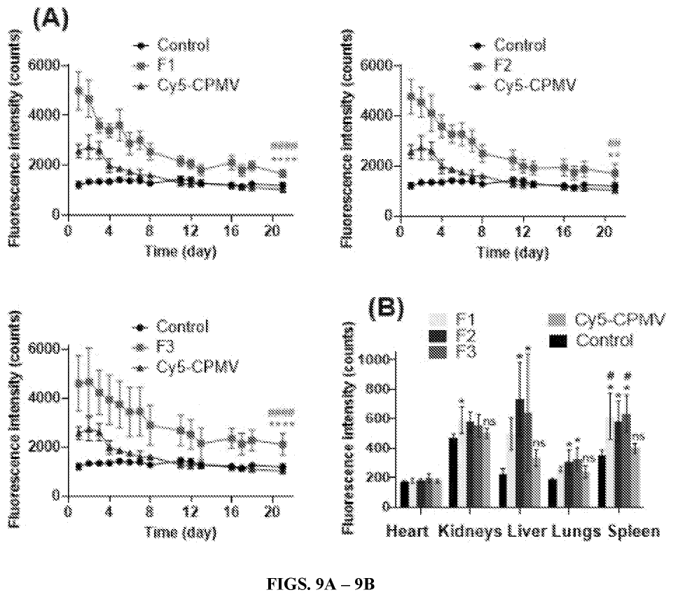

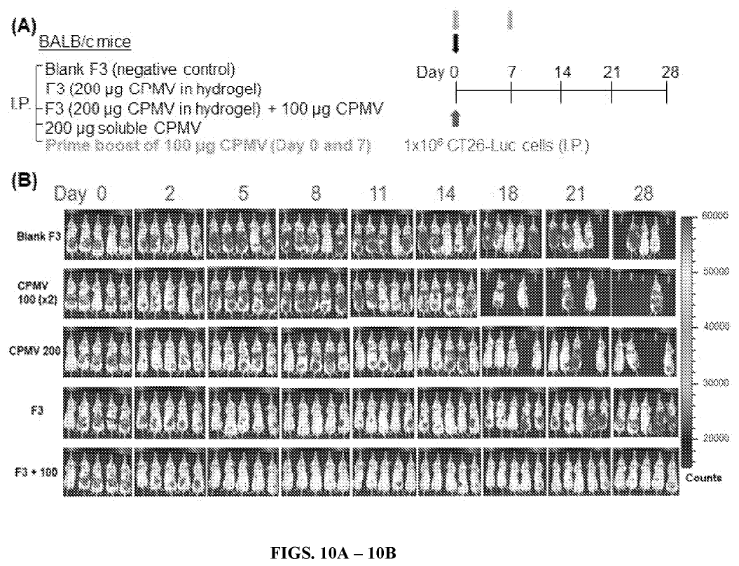

Characterization of Cy5-CPMV released from the hydrogels in vitro. Having established the potential for intermolecular interactions within the hydrogel, Applicant investigated whether the chemical reactivity of the matrix had a negative impact on nanoparticle stability. Cy5-CPMV particles released from the hydrogels on days 7 and 14 were characterized by native agarose gel electrophoresis, SDS-PAGE and TEM. The illumination of agarose gels with red light revealed Cy5 bands that matched the RNA signal under UV light and the protein bands under white light following staining with CBB (FIG. 9A). This confirmed the presence of intact particles containing all three components. Some particles remained in the loading wells, which may reflect particle aggregation or interactions with positively charged chitosan molecules affecting electrophoretic migration towards the anode. The chemical stability of Cy5-CP conjugates was confirmed by SDS-PAGE, which showed that the protein bands corresponding to the small and large CPs after staining with CBB appeared at the same positions as the fluorescent bands representing Cy5 (FIG. 9B). This confirmed that the covalent linkage between Cy5 and the particles remained stable after 14 days in the hydrogel matrix. Finally, the structural integrity of the Cy5-CPMV particles eluted from hydrogels was confirmed by TEM (FIG. 3). Taken together, these observations suggest that chemically modified CPMV nanoparticles are likely to maintain their particulate and molecular integrity following encapsulation within and release from the chitosan/GP hydrogels.

In vivo retention and release profiles. Cy5-CPMV-loaded formulations F1, F2 and F3 were injected subcutaneously behind the neck of shaved BALB/c mice to determine the retention and release profiles in vivo. Cy5-CPMV in PBS was injected as a control. The local retention of Cy5-CPMV was assessed over 21 days by fluorescence imaging of the injection site and ROI analysis. The signals from the single dose of soluble Cy5-CPMV decayed rapidly compared to the hydrogel formulations, disappearing almost completely by day 12 post-injection due to fast diffusion and clearance⁶³ (FIG. 4A). The signals from F1 and F2 lasted until day 18 and the signal from F3 was still present at the end of the experiment, indicating depot formation in situ followed by the slower diffusion of Cy5-CPMV from the injection site. Although the hydrogel significantly increased the residence time of CPMV, the excellent tissue residence time of the soluble formulation is also notable, probably reflecting the high stability of the CPMV nanoparticles. Quantitative fluorescence intensity analysis revealed that F3 was the only formulation that differed significantly from free Cy5-CPMV in terms of fluorescence decay (FIG. 4B). This agrees with the observed ability of F3 to outperform the other formulations in vitro (e.g., the shortest gelation time and slower release). Applicant also compared Cy5-CPMV local retention following subcutaneous injections of F3 (200 g single dose) versus soluble Cy5-CPMV (100 g every 14 days) and the outcome was intriguing. Bright fluorescence at the injection site was observed in both groups on day 15 but only in the F3 group on day 28, confirming the prolonged tissue residence due to depot formation (FIG. 4C). Although the reliability of fluorescence signals is limited by the potential for quenching or particle aggregation (especially in the confined subcutaneous injection site), the results nevertheless allowed us to compare the rate of Cy5-CPMV particle clearance when using soluble and slow-release formulations, supporting the enhanced local retention achieved by the administration of Cy5-CPMV in chitosan/GP hydrogels.⁷⁹

### Efficacy of 826-CPMV Loaded Hydrogel as a Single-Dose Vaccine.

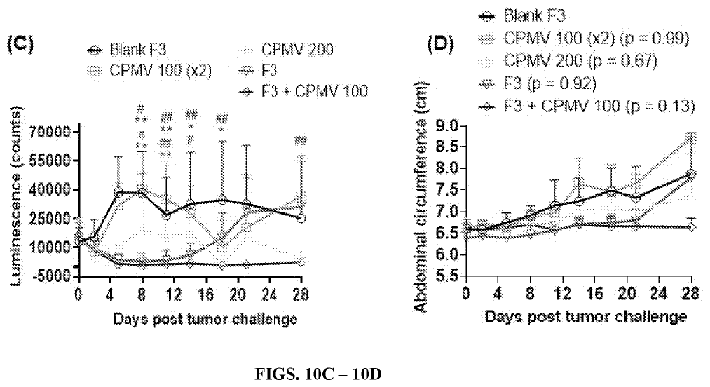

Bioconjugation of peptide epitope 826 to CPMV. Applicant conjugated the B-cell epitope 826 (peptide sequence 809-826 of the SARS-CoV-2 S protein) to CPMV using our two-step protocol as previously described.³⁹ This peptide is highly conserved and is not affected by the mutations that generated the Delta and Omicron variants of SARS-CoV-2 (FIG. 10). Applicant used NHS chemistry to attach the cross-linker SM(PEG)₄ to lysine side chains on CPMV (FIG. 5A). The resulting maleimide handles were quickly conjugated to the cysteine residues of peptide 826 in the presence of the polymer Pluronic F127, a surfactant used for peptide solubilization.⁷⁰ The 826-CPMV particles were purified by ultracentrifugation and characterized by SDS-PAGE, native agarose gel electrophoresis and TEM. SDS-PAGE revealed the presence of new CP bands with higher molecular weights than the native small and large CPs, reflecting the conjugation of the additional peptide (FIG. 5B). Quantitative analysis by densitometry indicated that each nanoparticle displayed ˜60 peptide epitopes, which is in agreement with our previous study.³⁹ Native agarose gel electrophoresis indicated that the 826-CPMV particles had a lower electrophoretic mobility than native CPMV, which can be attributed to the higher molecular weight and increase in hydrodynamic diameter (FIG. 5C). The presence of a higher-mobility band that appeared to be free RNA (stained with GelRed but not CBB) may indicate the release of RNA under the reaction conditions, in agreement with our previous work on the 826-CPMV formulation.⁷⁰ While some RNA is lost during the conjugation procedure, a significant amount of the RNA is retained within the formulation. Importantly RNA is not lost during hydrogel formulation (see FIG. 9). The structural integrity of 826-CPMV nanoparticles was confirmed by TEM, which revealed homogeneous icosahedral particles of ˜30 nm (FIG. 5D). Collectively, these data confirmed the synthesis of stable 826-CPMV nanoparticles for immunization studies.

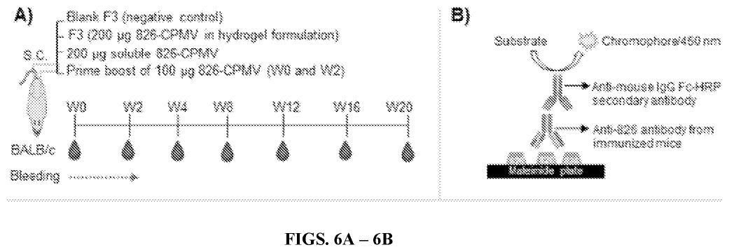

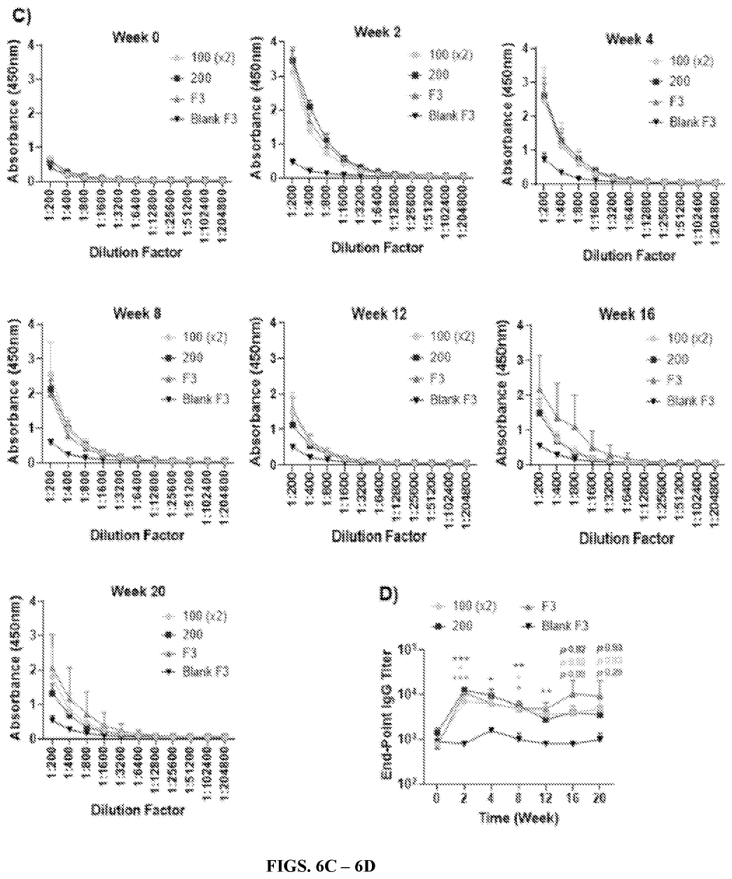

Immunogenicity of hydrogel F3 containing 826-CPMV particles. The immunogenicity of 826-CPMV formulated in chitosan/GP hydrogel F3 was evaluated in female BALB/c mice. Based on the previously reported dosing schedule for 826-CPMV,³⁹ a single dose of liquid formulation F3 containing 200 g of 826-CPMV particles was compared with the soluble particles in PBS administered as a single subcutaneous dose of 200 g or prime-boost doses of 100 g at the beginning of weeks 0 and 2 (FIG. 6A). Blood samples were collected by retro-orbital bleeding over 20 weeks and sera were screened for antibodies against epitope 826 by ELISA (FIG. 6B). The control group (F3 hydrogel without 826-CPMV particles) did not elicit antibodies, whereas all study groups produced anti-826 IgG (FIG. 6C). The injectable hydrogel formulation of 826-CPMV improved the antibody titers at later time points (between weeks 12 and 20) compared to the soluble formulation (FIG. 6D). Significantly high antibody concentrations were still apparent at week 20 following the administration of 826-CPMV particles in hydrogel F3. Differences in antibody titers were apparent at later time points with higher titers observed in animals immunized with 826-CPMV particles released from the F3 hydrogel vs single administration of 200 g of 826-CPMV particles or prime-boost with 100 g of 826-CPMV particles (FIG. 6C, FIG. 6D). This is consistent with the prolonged tissue residence time and slow release of CPMV from the injectable hydrogel compared to the faster clearance of the soluble CPMV formulation (FIG. 4). The data provide further evidence that intact and biologically active CPMV nanoparticles released from the hydrogel retained their biological properties, supporting the in vitro stability data (FIG. 3, FIG. 9). The chitosan/GP slow-release technology is therefore highly compatible with plant virus nanotechnology. Our results are important because many nations have now initiated repeat vaccinations with shorter intervals in an attempt to control COVID-19, whereas a slow-release formulation could provide long-lasting immunity by creating a depot that releases vaccine antigens over a period of several months. The use of such formulations would therefore alleviate some of the burden on global health systems by reducing the number of vaccination appointments needed to achieve population-wide protection.

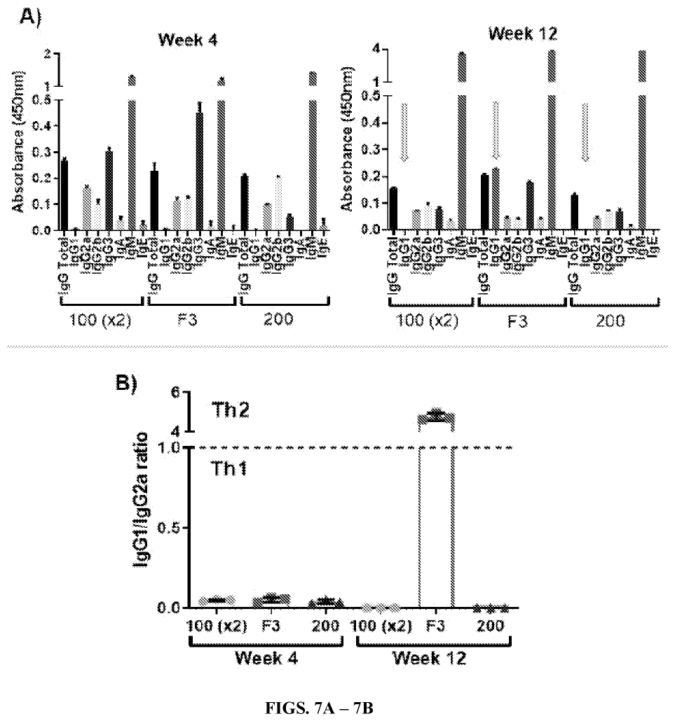

Antibody isotyping. Finally, Applicant analyzed the Ig isotypes and IgG subclasses in plasma from weeks 4 and 12 and thus reveal whether hydrogel vaccine F3 induced a Th1-biased response (IgG1/IgG2a ratio <1) or a Th2-biased response (IgG1/IgG2a ratio >1). Th1 cells produce cytokines such as interferon γ (IFN-γ) that instruct B cells to produce opsonizing antibodies (IgG2a/b) and stimulate macrophages for phagocytic activity against intracellular pathogens (e.g., viruses). In contrast, Th2 cells produce interleukin 4 (IL-4) that instructs B cells to secrete neutralizing antibodies (IgG1) for humoral protection against pathogens or toxins in the extracellular environment.⁴⁰ Applicant observed comparable Ig isotype profiles in all groups at week 4, but evident differences at week 12 due to IgG1 becoming exclusively prominent in the F3 group (arrows in FIG. 7A). Based on the IgG1/IgG2a ratio, Applicant found that F3 induced a Th1-biased response at week 4 but shifted to a Th2-biased response at week 12, while the immune response for the soluble 826-CPMV groups remained Th1-biased throughout the experiment (FIG. 7B). CPMV-based vaccines were previously shown to induce Th1-biased responses against cancers,^{41,80,81} but Th2-biased responses at later time points have been reported for other shared epitopes from SARS-CoV and SARS-CoV-2 S protein, reflecting a shift from Th1 typically after the second boost injection.⁴³ The Th1/2 response was deemed to be dependent on the SARS-CoV2 S protein epitope.³⁹⁴³ With regard to epitope 826, Applicant and others³⁹ observed only Th1-biased responses for soluble 826-CPMV administered using the prime-boost schedule, which implies that the observed shifting bias in the F3 group from Th1 to Th2 is possibly due to the immune-enhancing adjuvant capability of chitosan^{54-56,82} and/or the slow-release characteristics of the hydrogel F3. The first CPMV nanoparticles released from the gel can diffuse through lymph vessel pores and find their way to the lymph node, where they interact directly with B cells to induce immediate IgG2a production (Th1 bias) without prior interactions with T cells.^{40,83} However, longitudinal and delayed release may induce more Th2 bias because the particles are likely to interact with antigen presenting cells due to their prominent recognition by pre-existing opsonizing antibodies.⁴⁶ The comparative release profiles of soluble particles versus hydrogels may help to determine whether CPMV-based vaccines are inherently Th1-mediated adjuvants, or whether the nature of the epitope is the main determinant of Th1/2 bias.

Vaccine efficacy and safety are important design parameters and while Th2 bias is desired to elicit neutralizing IgG1 antibodies for humoral protection against viruses prior to cell entry and establishment of infection, reports highlight risk of antibody dependent enhancement (ADE) with the SARS and Middle East Respiratory Syndrome (MERS) coronaviruses vaccine candidates.^{84,85} Some reports suspected similar risk of ADE for SARS-CoV-2 vaccines;^{86,87} nevertheless, the rationale design and choice of target epitope may provide greater safety compared to subunit vaccines containing RBD or full-length S protein.

### Conclusion

Applicant have formulated an injectable hydrogel containing CPMV conjugated to B-cell epitope 826 as a single-dose vaccine candidate for COVID-19. CPMV hydrogel formulations were prepared using chitosan and GP solutions to yield a liquid mixture that was homogenized with CPMV particles at room temperature. HMW chitosan formulations (F3) containing 0-4.5 mg mL⁻¹ CPMV achieved a relatively fast transition from liquid solutions to gels at 37° C. (gelation time 5-8 min), and slowly released Cy5-CPMV particles in vitro and in vivo. Most importantly, F3 containing CPMV labeled with epitope 826 from the SARS-CoV-2 S protein induced high antibody titers over 20 weeks, with an associated shift from Th1-biased to Th2-biased profiles. These findings show that CPMV nanoparticles can be effectively formulated in chitosan/GP hydrogels, and are released over several months as intact and biologically active particles with conserved immunotherapeutic efficacy. The proposed formulation not only represents a single-dose vaccine candidate to address future pandemics, but also facilitate the development of long-lasting plant virus-based nanomedicines for diseases that require long-term treatment.

### Experiment No. 2

Cancer is the leading cause of death worldwide, and colon cancer represents the second leading cause of cancer-related deaths and the third most common cancer in terms of new cases.⁸⁸ In 2020, there were 935,000 deaths and 1.93 million cases of colon cancer globally.⁸⁹ These statistics are enormous considering limited accessibility to healthcare setting patients may have faced over the last two years due to the coronavirus diseases 2019 (COVID-19) pandemic.⁹⁰ Poor cancer screening combined with ineffective control of risk factors and treatment limitations are among the factors that worsen cancer mortality and morbidity globally, especially in developing countries.⁹¹ The mainstay treatment for colon cancer is laparoscopic surgery combined with preoperative or postoperative adjuvant chemotherapy as well as radiotherapy.⁹² However recurrence occurs at a rate of 70% within 2 years and 90% within 5 years after surgery.⁹³ Therefore, there is need to develop treatments to prevent recurrence.

Over the last two decades, immunotherapies have shown some success against metastatic colon cancer; with clinical approval of monoclonal antibodies such as cetuximab and panitumumab, which are anti-epidermal growth factor receptor (EGFR) antibodies.⁹⁴ In the preclinical setting, Applicant have shown that in situ vaccination immunotherapy using immunostimulatory cowpea mosaic virus (CPMV) nanoparticles is a promising strategy for colon cancer therapy: CPMV is a 30 nm-sized icosahedral, non-glycosylated plant virus with a bipartite RNA genome that—while noninfectious toward mammals—triggers potent innate immune activation through recognition by pattern recognition receptors (PRRs).⁹⁵ Applicant have demonstrated that CPMV elicits potent systemic and durable anti-tumor immunity through priming the innate immune system; efficacy has been reported in mouse models of colon cancer,^{96,97} as well as other tumor models^{98,99} and canine patients.¹⁰⁰ In recent work, Applicant also demonstrated that CPMV targeted to the lungs primes innate immune activation and therefore protects from onset of lung metastasis.¹⁰¹

Based on this body of data, Applicant hypothesized that intraperitoneal (IP) administration of CPMV could protect onset of metastatic colon tumor growth. Applicant tested this using a mouse model with IP challenge of CT26 colon cancer cells; CPMV was administered as soluble injectable in buffer as well as slow-release hydrogel formulation. In prior work, when used as treatment, Applicant found that prime-boost administrations are needed to achieve potent efficacy, and that the need for repeated administration can be alleviated through formulation as slow-release injectable.¹⁰² From a clinical point of view, repeated IP administrations are not ideal given the hospitalization requirements for IP injections,¹⁰³ which would increase the management cost and worsen patient's quality of life—already impaired by the consequences of colon cancer disease (such as abdominal pain, change in bowel movements, blood loss and anaemia, fatigue, and weight loss).⁹² Then, Applicant used CPMV crystals assembled from CPMV and dendrimers¹⁰²; the self-assembled CPMV crystals act as depot and extend the retention time within the IP space—therefore leading to prolonged boosts and potent efficacy after single administration. However, the PAMAM dendrimers are known for dose limiting toxicity^{104,105} that may negatively impact the reputed translational potential of this approach. Thus, there is need to advance the formulation chemistry, and therefore here Applicant turned toward the formulation of injectable hydrogels. The purpose of the present study was to use the biocompatible chitosan polymer as a scaffold technology for slow-release formulation of CPMV-based immunotherapeutics.

Chitosan is a family of linear polysaccharides made up of diverse amounts of glucosamine residues produced by deacetylation of the biopolymer chitin.¹⁰⁸ Owing to its excellent biocompatibility and biodegradability, chitosan was initially approved as a generally regarded as safe (GRAS) excipient in 2002 by the European Pharmacopeia 6.0,¹⁰⁸ and 10 years later by the 29th edition of the United States Pharmacopeia (USP) 34-NF.¹¹⁰ Chitosan is currently part of the core excipients in many marketed pharmaceutical formulations such as BST-CarGel™ intended for cartilage regeneration.¹¹¹ Chitosan's cationic character is responsible for most of the attractive pharmaceutical properties of the polymer, including controlled release behaviour, mucoadhesion, transfection, permeation enhancement as well as in situ gelation.¹⁰⁸ Chitosan-based in situ gelling formulations are prepared by mixing protonated chitosan with the anionic β-glycerophosphate (GP) salt; the resultant solution appears fluid at or below room temperature but turns into a gel at physiological temperature (37° C.). The thermo-sensitivity of chitosan/GP mixture arises from multiple interactions between GP anions and polycationic chitosan chains; including electrostatic binding and increased proton transfer at higher temperature, which leads to reduced chitosan charge density and polymer chains rapprochement causing gel formation due to hydrophobic inter chain bonding.^{112,113} The in situ forming chitosan/GP hydrogels have shown some success as a formulation technology for various biomedical applications, such as local drug delivery for cancers;^{112,114} tissue engineering,¹¹⁵ and controlled release of nanoparticles.^{116,117}

In this study, Applicant formulated CPMV nanoparticles in chitosan/GP hydrogels for single dose IP administration with the goal to prevent colon cancer growth. Applicant assessed the slow-release capability of the hydrogel formulation in the IP space using CPMV labeled with the fluorophore sulfo-cyanine 5 (Cy5)-enabling longitudinal imaging. Finally, Applicant tested the antitumor efficacy of a single dose CPMV-in-hydrogel (alone or co-injected with soluble CPMV) versus single and multiple IP injections of soluble CPMV particles using colon cancer models before disease establishment.

### CPMV Production and Modification

CPMV was propagated in and purified from cowpea plants (Vigna unguiculata). For imaging studies, Cy5-labelled CPMV was synthesized, and both CPMV and Cy5-CPMV particles were characterized by UV-vis spectroscopy, sodium dodecylsulfate polyacrylamide gel electrophoresis (SDS-PAGE), size exclusion chromatography (SEC) and dynamic light scattering (DLS). All protocols were previously described,^{118,119} and detailed methods are provided in the supporting information (SI) file.

### Hydrogel Preparation and Characterization

Liquid formulations were prepared by mixing a hydrochloric solution of chitosan with the aqueous solution of sodium glycerophosphate at room temperature, and CPMV or Cy5-CPMV particles were dispersed in the resultant fluid by vortex-mixing. Key formulations properties, such as gelation time, gel swelling, degradation and release profiles were determined as previously reported.¹²⁰ The particulate characteristics of in vitro released CPMV particles were investigated by SEC and DLS to confirm particles integrity. Detailed procedures of the hydrogel preparation and characterization are described in the SI file.

### Animal Studies

Ethical statements. All animal procedures were performed following the guidelines of the Institutional Animal Care and Use Committee (IACUC) of the University of California San Diego (UCSD), using the protocols approved by the UCSD's Animal Ethics committee. Applicant used 7-8 weeks old BALB/c female mice from the Jackson Laboratory (Bar Harbor, ME, USA) and hosted animals at the UCSD Moores Cancer Center with full access to food and water.

In vivo release of Cy5-CPMV from hydrogels. Applicant injected intraperitoneally 100 μL of F1-F3 liquid formulations containing 450 g Cy5-CPMV or soluble 450 g Cy5-CPMV in 100 μL PBS to belly shaved healthy mice on day 0 (5/group). Applicant maintained animals on an alfalfa-free diet 1 week before particles injections and later throughout the study. At predefined time intervals, the IP space was imaged using a Xenogen IVIS 200 Optical Imaging System (Caliper Life Sciences, Hopkinton, MA, USA) and IVIS software was used for quantitative analysis of fluorescence using the region of interest (ROI). On day 21 mice were sacrificed; and major organs (kidneys, liver, lungs, spleen, and heart) were collected for Cy5 fluorescence measurement as above, and fluorescence intensity was assessed using ROI.

Colon cancer prevention. CT-26-Luciferase cells (CT-26-Luc cells) were cultured in ATCC-formulated RPMI-1640 medium supplemented with 10% v/v fetal bovine serum+antibiotics, and maintained at 37° C. in a 5% C02 incubator. CT-26-Luc cells (1×106 in 150 μL PBS/mouse) were intraperitoneally injected into female BALB/c mice. Five treatments were tested (n=5 mice per group): immediately after tumor inoculation, (i) soluble CPMV 200 g (150 μL PBS, single dose) and (ii) 100 μg (150 μL PBS, two weekly injections) were i.p. injected to mice separated in two groups (group CPMV 200 and CPMV 100 (×2)). For hydrogel formulations, (iii) F3 containing CPMV 200 μg/150 μL or (iv) hydrogel without CPMV (blank F3) were injected 30 min before cell inoculation to allow gel formation and avoid cell entrapment in the hydrogel matrix. To prevent any lag phase effects, (v) an additional animal group was i.p. treated with F3 and further injected with soluble CPMV 100 μg/150 μL PBS immediately after tumor cells inoculation. Tumour growth was monitored every 2/3 days using bioluminescence IVIS imaging (5 min post-injection of luciferin 15 mg mL⁻¹/150 μL PBS, with a 3 min exposure time) and abdominal circumference measurements. Bioluminescence intensity was calculated using the ROI analysis.

### Statistical Analysis

All data were processed and analyzed using GraphPad Prism v9.0.2 (GraphPad Software, San Diego, CA, USA). Comparative analysis was performed by one-way analysis of variance (ANOVA) followed by Tukey's multiple comparison test. Asterisks in the figures indicate statistical differences between a study group and the control (*p<0.05; **p<0.01; ***p<0.001; ****p<0.0001), while hashtags mark statistical differences between study groups (#p<0.05; ##p<0.01; ###p<0.001)

### Results and Discussion

### CPMV/Cy5-CPMV Nanoparticles Production

CPMV is a plant virus member of the genus comovirus in the family Comoviridae. It consists of 60 copies each of small (24 kDa) and large (41 kDa) coat protein (CP) units that self-assemble into an icosahedral particle encapsulating the viral genome composed of two molecules of positive-strand RNA (RNA-1 and RNA-2).¹²¹ The proteinaceous nature of CPMV particles allows multivalent modifications through surface-exposed amino acid handles to impart useful functionalities such as bioimaging capabilities.¹²² Using previously published preparation protocols, Applicant produced CPMV particles 55 mg from infected leaves 100 g of V. unguiculata, and found the 260/280 nm absorbance ratio of 1.75, which is indicative of good particle purity.¹¹⁸ To produce fluorescent nanoparticles for in vivo release studies, CPMV particles were labeled with the fluorophore Cy5 using the NHS-activated method to target solvent-exposed Lysine side chains and the conjugated was characterized using several techniques (FIG. 8). The UV-Vis spectrum of purified Cy5-CPMV particles exhibited the spectral bands characteristic to the UV-Vis absorption features of both the viral capsid protein at 260 nm and the Cy5 dye at 647 nm; Applicant determined that ˜30 dyes per CPMV were displayed, previously reported to be ideal for fluorescence bioimaging.¹²² Covalent CPMV modification with Cy5 was confirmed by SDS-PAGE showing that both the small and large protein were labeled with the dye. Lastly, particle integrity was also confirmed by SEC, which showed a single peak indicating that Cy5-CPMV's viral components (i.e., CP detected at 260 nm and RNA at 280 nm) co-eluted with the Cy5 dye (detected at 647 nm) at the same solvent elution volume (11.5 mL) as for CPMV wild type, underlining particles intactness since there were no other prominent peaks for aggregates, free proteins, or unconjugated dyes. The synthesized Cy5-CPMV particles were used for in vivo evaluation of hydrogel release in the peritoneum.

### Hydrogel Properties

The thermo-sensitivity is the unique property that enables injectable hydrogels to meet key requirements for effective locoregional delivery of therapies:¹²³ (i) be in fluid state at room temperature to allow drug incorporation, injection through a small size needle (>23 G), with low viscosity (<1 Pa·s); (ii) be able to undergo sol-to-gel transition at physiological temperature (37° C.) to enable strong depot formation, avoiding dilution in body fluids, and achieve slow but sustained drug release for prolonged efficacy.

To prepare CPMV-in-chitosan/GP formulations, GP solution (50%) was added to a particular acidic solution of chitosan (2.2%), and, after homogenization, the resultant liquid product was vortex-mixed with CPMV solution at room temperature but form a gel after incubation at 37° C. (FIG. 8). Irrespective of the type of VNPs (CPMV or Cy5-CPMV) or their absence, the formulations composed of high molecular weight (MW) chitosan exhibited the shortest gelation times (5-8 min), while the gelation times for medium MW chitosan formulations were intermediate (8-15 min) and gelation times of low MW chitosan-based formulations were the longest (11-18 min). Formulations composed of low, medium and high MW chitosan were subsequently named F1, F3, and F3, respectively. The injectability of these formulations was realized by passage through 28 G, 27 G, and 26 G needles for F1, F2 and F3, respectively. This ranking is consistent with the viscosity of these liquid formulations Applicant previously recorded at 25° C. (0.099 Pa·s for F1; 0.202 Pa·s for F2; and 0.482 Pa·s for F3).¹²⁰ The formulation F3 was found to be the most viscous, but the recorded viscosity value (<1 Pa·s) and the passage through a 26 G needle suggest F3 formulation has huge potential for good flow and spreading within the TME;¹²³ other authors established the injectability of a chitosan/GP-based formulation by passing through a 23 G needle.¹²⁴

Applicant used Cy5-CPMV particles to characterize hydrogel degradation, swelling and release profiles; these data are reported in Nkanga et al.¹²⁰ Although no obvious gel degradation or swelling was observed, the UV spectrometric analysis of the release medium revealed continuous release of CPMV particles from the formulated hydrogels. The release rate of CPMV from all the hydrogels was much slower (only 20% released by day 28) compared to the diffusion rate of CPMV particles in the PBS medium (up to 95% depleted by day 10). The fast diffusion of CPMV particles in solution is essentially governed by the Brownian motion, whereas the slow release of CPMV from the hydrogels may reflect the existence of physical or/and chemical hindrances within the polymeric matrix;^{125,126} thus Applicant characterized CPMV particles from hydrogels to verify whether their properties remained unchanged (FIG. 9).

The particles size analysis by DLS indicated that the release media from all the hydrogels contained particles with size distribution patterns similar to those recorded on soluble/free CPMV and CPMV wild type samples (FIG. 9A). SEC revealed the presence of intact CPMV particles in all the samples analyzed up to day 14, with the viral components co-eluting at the same solvent elution volume as CPMV incubated in PBS as well as fresh CPMV wild type. FIG. 9B compares the chromatographic profiles of CPMV coat protein (CP) detected at 260 nm and indicates that the 260:280 nm absorbance ratios remained within the range acceptable for good particles purity (1.7-1.8),¹¹⁸ highlighting that the CP to RNA ratios were maintained throughout the incubation period, which is an evidence of particle integrity. Collectively, these data verified the stability of CPMV longitudinally released from the hydrogels, which is a commendable asset because the antitumor immunotherapeutic efficacy is contingent on CPMV particulate characteristics.^{127,128}

### In Vivo Slow-Release and Biodistribution of Cy5-CPMV

To assess the hydrogel local retention in the IP space, healthy BALB/c mice were intraretinally injected with a single dose of 450 g Cy5-CPMV either dissolved in PBS or dispersed in chitosan/GP liquid formulations made up of low, medium, and high molecular weight chitosan (also denoted F1, F2, and F3, respectively). Mice were imaged over 21 days post-injection and sacrificed at endpoint (day 21) for fluorescence analysis of major organs. Applicant observed that soluble Cy5-CPMV particles cleared faster than hydrogel formulated particles: the free Cy5-CPMV group showed no signal from day 7 on, while all the hydrogels (F1-F3) groups exhibited fluorescence until day 21 (FIG. 9A). The longitudinal assessment of fluorescence intensity in the IP space revealed a drastic decay for soluble particles while the signals from all the hydrogels decreased gradually, indicating that the encapsulated particles can diffuse through the polymer matrix and describe sustained release profiles. From day 7 on, no significant difference was observed between soluble Cy5-CPMV group and untreated animals (control), whereas all F1-F3 groups were significantly different from the control on day 21. When compared to both the control and soluble particles, the fluorescence intensity due to F1 and F3 showed p<0.0001 while the difference with F2 yielded p<0.01, suggesting that the retention time was not contingent on chitosan molecular weight. Based on its short gelation time and extended-release effect, the hydrogel formulation F3 was selected for antitumor efficacy testing (discussed in the next section). Overall, all the hydrogel formulations demonstrated 3-fold longer residence time (>21 days) in the peritoneum than soluble particles (<7 days), which clearly establishes the slow-release capabilities of chitosan/GP formulations due to depot effects. It is worth mentioning that the fluorescence intensity may be underestimated given the dynamic environment and motion within the peritoneum. In addition, fluorescence quenching may occur. Nonetheless, the observed local retention is commendable based on the previously reported data for other slow-release systems, such as the thermosensitive polycaprolactone-poly(ethylene glycol)-based hydrogels¹²⁹ and polyamidoamine (PAMAM) dendrimers,¹⁰² which respectively achieved 8 and 14 days retention time following IP injections. The fluorescence analysis of organs harvested on day 21 showed no detectable particles in the group injected with soluble particles (FIG. 10, FIG. 9B), while hydrogels groups exhibited remarkable fluorescence signals in the reticulum endothelial system (RES) organs (liver, spleen, kidneys and lungs), a typical biodistribution profile for CPMV particles.^{130,131} This further corroborates sustained release and prolonged retention of CPMV when formulated as hydrogel.

### Tumor Prevention

The antitumor prophylactic efficacy of CPMV in chitosan/GP hydrogel (F3) was assessed in an IP disseminated colon cancer model (CT-26). Applicant inoculated Luc-expressing CT26 cells to BALB/c mice and immediately randomized them to one of the following intraperitoneal treatments (FIG. 10A): (i) single dose blank F3 (hydrogel without CPMV, negative control); (ii) double weekly dose of 100 g CPMV in PBS (day 0 and 7); (iii) single dose of 200 g CPMV in PBS; (iv) single dose of 200 g CPMV in hydrogel (F3); and (v) single dose of 200 μg CPMV in hydrogel (F3)+100 μg CPMV in PBS, injected within a 30 min window to allow complete gelation prior to free particle injection, avoiding biased additional particles entrapment in the gel. Applicant monitored tumor growth using bioluminescence imaging and abdominal circumference measurements over 28 days. The model and experimental design was chosen to reflect low tumor burden after surgical debulking; Applicant specifically asked whether CPMV treatment could protect animals from onset of tumor growth.

Total luminescence in mice treated with F3+CPMV 100 g was statistically lower than total luminescence observed in mice treated with 2×CPMV 100 g or blank F3 throughout the study period (FIG. 10B, FIG. 10C), indicating excellent tumor growth inhibitory effect of the proposed combination (F3+CPMV 100 μg) enabling immediate and sustained immune activation through soluble and slowly released CPMV. Mice treated with F3 only also showed reduced luminescence over the first two weeks of the study, but sudden increase was observed at later time points. This is likely because the CT-26 cells form a fast-growing mouse tumor,¹³⁴ underestimating the number of particles being slowly released from the hydrogel; thus, Applicant anticipated an additional dose (CPMV 100 g) to supplement the F3 slow-release behavior. This approach is often adopted when dealing with slow release formulations that exhibit lag phase, as it is the case for the branded depot Risperdal Consta™, the marketed poly(lactide-co-glycolide) (PLGA) microspheres of risperidone.¹³⁵ Consistent with luminescence measurements, data from belly circumference variations indicated that F3+CPMV 100 μg demonstrated the most potent efficacy against tumor establishment compared to any other group (FIG. 10D): an increase in belly circumference throughout the study was evident. This establishes the combination F3+CPMV 100 μg as a promising antitumor prophylactic treatment for colon cancer.

### Conclusion

In this study, Applicant assessed the antitumor preventative efficacy of CPMV in injectable hydrogels made up of chitosan and glycerophosphate. The hydrogel formulation loaded with fluorescent cyanine 5 labeled CPMV exhibited local retention in the intraperitoneal (IP) space and CPMV released from the hydrogel was detectable over 3 weeks—in stark contrast soluble CPMV was cleared within one week post administration. Using a colon cancer mouse model, Applicant demonstrate that combined soluble and hydrogel-formulated CPMV is potent to prevent onset of tumor growth. Codelivery of F3 (CPMV-in-hydrogel) and soluble CPMV 100 μg significantly inhibited the CT26 cell growth over 28 days post tumor cell inoculation and treatment, while soluble single or double dose of soluble particles failed to prevent tumor growth. The observed antitumor efficacy confirms that CPMV released from hydrogels retained their inherent immunogenicity and highlights the potential of hydrogel and slow-release formulations to improve colon cancer therapy through extended local retention, making the long-acting single dose treatment possible to improve patient's quality of life. Plant virus-in-hydrogels are promising for treatment of colon cancer immunotherapies; in particular in the setting of adjuvant therapy post-surgery.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Zhu, N.; Zhang, D.; Wang, W.; Li, X.; Yang, B.; Song, J.; Zhao, X.; Huang, B.; Shi, W.; Lu, R.; Niu, P.; Zhan, F.; Ma, X.; Wang, D.; Xu, W.; Wu, G.; Gao, G. F.; Tan, W. A Novel Coronavirus from Patients with Pneumonia in China, 2019. N. Engl. J. Med. 2020, 382 (8), 727-733. https://doi.org/10.1056/nejmoa2001017.
2. World Health Organization. The True Death Toll of COVID-19. Https://Www.Who.Int/Data/Stories/the-True-Death-Toll-of-Covid-19-Estimating-Global-Excess-Mortality (Last Accessed 1 Dec. 2021).
3. Statista. Number of Cumulative Cases of Coronavirus (COVID-19) Worldwide from Jan. 22, 2020 to Nov. 22, 2021, by Day. Https://Www.Statista.Com/Statistics/1103040/Cumulative-Coronavirus-Covid19-Cases-Number-Worldwide-by-Day/. (Last Accessed on 1 Dec. 20.
4. Polack, F. P.; Thomas, S. J.; Kitchin, N.; Absalon, J.; Gurtman, A.; Lockhart, S.; Perez, J. L.; Pérez Marc, G.; Moreira, E. D.; Zerbini, C.; Bailey, R.; Swanson, K. A.; Roychoudhury, S.; Koury, K.; Li, P.; Kalina, W. V.; Cooper, D.; Frenck, R. W.; Hammitt, L. L.; Türeci, Ö.; Nell, H.; Schaefer, A.; Ünal, S.; Tresnan, D. B.; Mather, S.; Dormitzer, P. R.; Şahin, U.; Jansen, K. U.; Gruber, W. C. Safety and Efficacy of the BNT162b2 MRNA Covid-19 Vaccine. N. Engl. J. Med. 2020, 383 (27), 2603-2615. https://doi.org/10.1056/nejmoa2034577.
5. Merryn Voysey, Sue Ann Costa Clemens, Shabir A Madhi, Lily Y Weckx, Pedro M Folegatti, Parvinder K Aley, Brian Angus, V. L. B.; Shaun L Barnabas, Qasim E Bhorat, Sagida Bibi, Carmen Briner, Paola Cicconi, Andrea M Collins, Rachel Colin-Jones, C. L. C.; Thomas C Darton, Keertan Dheda, Christopher J A Duncan, Katherine R W Emary, Katie J Ewer, Lee Fairlie, Saul N Faust, S. F.; Daniela M Ferreira, Adam Finn, Anna L Goodman, Catherine M Green, Christopher A Green, Paul T Heath, Catherine Hill, Helen Hill, I. H.; Susanne H C Hodgson, Alane Izu, Susan Jackson, Daniel Jenkin, Carina C D Joe, Simon Kerridge, Anthonet Koen, Gaurav Kwatra, R. L.; Alison M Lawrie, Alice Lelliott, Vincenzo Libri, Patrick J Lillie, Raburn Mallory, Ana V A Mendes, Eveline P Milan, A. M. M.; Alastair McGregor, Hazel Morrison, Yama F Mujadidi, Anusha Nana, Peter J O'Reilly, Sherman D Padayachee, Ana Pittella, E. P.; Katrina M Pollock, Maheshi N Ramasamy, Sarah Rhead, Alexandre V Schwarzbold, Nisha Singh, Andrew Smith, Rinn Song, M. D. S.; Eduardo Sprinz, Rebecca K Sutherland, Richard Tarrant, Emma C Thomson, M Estee Torok, Mark Toshner, David P J Turner, J. V.; Tonya L Villafana, Marion E Watson, Christopher J Williams, Alexander D Douglas, Adrian V S Hill, Teresa Lambe, Sarah C Gilbert, Andrew J Pollard, on behalf of the O. C. V. T. G. Safety and Efficacy of the ChAdOx1 NCoV-19 Vaccine (AZD1222) against SARS-CoV-2: An Interim Analysis of Four Randomised Controlled Trials in Brazil, South Africa, and the UK. Lancet 2021, 397 (10269), 99-111. https://doi.org/10.1016/S0140-6736(20)32661-1.
6. GOV.UK. Moderna COVID-19 Vaccine Authorised by UK Medicines Regulator. Jan. 8, 2021. Https://Www.Gov.Uk/Govemment/News/Moderna-Covid-19-Vaccine-Authorised-by-Uk-Medicines-Regulator (Accessed Dec. 30, 2021).
7. World Health Organization. WHO Coronavirus (COVID-19) Dashboard. Https://Covid19.Who.Int/(Last Accessed 30 Dec. 2021).
8. Moore, S.; Hill, E. M.; Tildesley, M. J.; Dyson, L.; Keeling, M. J. Vaccination and Non-Pharmaceutical Interventions for COVID-19: A Mathematical Modelling Study. Lancet Infect. Dis. 2021, 21 (6), 793-802. https://doi.org/10.1016/S1473-3099(21)00143-2.
9. Chung, Y. H.; Church, D.; Koellhoffer, E. C.; Osota, E.; Shukla, S.; Rybicki, E. P.; Pokorski, J. K.; Steinmetz, N. F. Integrating Plant Molecular Farming and Materials Research for Next-Generation Vaccines. Nat. Rev. Mater. 2021, 0123456789. https://doi.org/10.1038/s41578-021-00399-5.
10. Matrajt, L.; Eaton, J.; Leung, T.; Dimitrov, D.; Schiffer, J. T.; Swan, D. A.; Janes, H. Optimizing Vaccine Allocation for COVID-19 Vaccines Shows the Potential Role of Single-Dose Vaccination. Nat. Commun. 2021, 12 (1), 1-18. https://doi.org/10.1038/s41467-021-23761-1.
11. Tuite, A.; Zhu, L.; Fisman, D.; Salomon, J. Alternative Dose Allocation Strategies to Increase Benefits From Constrained COVID-19 Vaccine Supply. Ann. Intern. Med. 2021, 174 (4), 570-572.
12. Barnabas, R. V. A. W. A Public Health COVID-19 Vaccination Strategy to Maximize the Health Gains for Every Single Vaccine Dose. Ann Intern Med. 2021, 174(4), 552-553. https://doi.org/doi:10.7326/M20-8060.
13. Paltiel, A. D.; Zheng, A.; Schwartz, J. L. Speed versus Efficacy: Quantifying Potential Tradeoffs in COVID-19 Vaccine Deployment. Ann. Intern. Med. 2021, 174 (4), 568-570. https://doi.org/10.7326/M20-7866.
14. Sabino, E. C.; Buss, L. F.; Carvalho, M. P. S.; Prete, C. A.; Crispim, M. A. E.; Fraiji, N. A.; Pereira, R. H. M.; Parag, K. V.; da Silva Peixoto, P.; Kraemer, M. U. G.; Oikawa, M. K.; Salomon, T.; Cucunuba, Z. M.; Castro, M. C.; de Souza Santos, A. A.; Nascimento, V. H.; Pereira, H. S.; Ferguson, N. M.; Pybus, O. G.; Kucharski, A.; Busch, M. P.; Dye, C.; Faria, N. R. Resurgence of COVID-19 in Manaus, Brazil, despite High Seroprevalence. Lancet 2021, 397 (10273), 452-455. https://doi.org/10.1016/S0140-6736(21)00183-5.
15. Davies, N. G.; Abbott, S.; Barnard, R. C.; Jarvis, C. I.; Kucharski, A. J.; Munday, J. D.; Pearson, C. A. B.; Russell, T. W.; Tully, D. C.; Washburne, A. D.; Wenseleers, T.; Gimma, A.; Waites, W.; Wong, K. L. M.; van Zandvoort, K.; Silverman, J. D.; Diaz-Ordaz, K.; Keogh, R.; Eggo, R. M.; Funk, S.; Jit, M.; Atkins, K. E.; Edmunds, W. J. Estimated Transmissibility and Impact of SARS-CoV-2 Lineage B.1.1.7 in England. Science (80-.). 2021, 372 (6538). https://doi.org/10.1126/science.abg3055.
16. Callaway, E. DELTA CORONAVIRUS VARIANT: SCIENTISTS BRACE FOR IMPACT. Nature 2021, 595, 17-18.
17. Challen, R.; Brooks-Pollock, E.; Read, J. M.; Dyson, L.; Tsaneva-Atanasova, K.; Danon, L. Risk of Mortality in Patients Infected with SARS-CoV-2 Variant of Concern 202012/1: Matched Cohort Study. BMJ 2021, 372, 1-10. https://doi.org/10.1136/bmj.n579.
18. Chen, Y.; Zuiani, A.; Fischinger, S.; Mullur, J.; Atyeo, C.; Travers, M.; Lelis, F. J. N.; Pullen, K. M.; Martin, H.; Tong, P.; Gautam, A.; Habibi, S.; Bensko, J.; Gakpo, D.; Feldman, J.; Hauser, B. M.; Caradonna, T. M.; Cai, Y.; Burke, J. S.; Lin, J.; Lederer, J. A.; Lam, E. C.; Lavine, C. L.; Seaman, M. S.; Chen, B.; Schmidt, A. G.; Balazs, A. B.; Lauffenburger, D. A.; Alter, G.; Wesemann, D. R. Quick COVID-19 Healers Sustain Anti-SARS-CoV-2 Antibody Production. Cell 2020, 183 (6), 1496-1507.e16. https://doi.org/10.1016/j.cell.2020.10.051.
19. Roberto, B. E. J. T. Assessing the Human Immune Response to SARS-CoV-2 Variants. Nat. Med. 2021, 27 (4), 568-569. https://doi.org/10.1038/s41591-021-01240-w.
20. Wang, Z.; Schmidt, F.; Weisblum, Y.; Muecksch, F.; Barnes, C. O.; Finkin, S.; Schaefer-Babajew, D.; Cipolla, M.; Gaebler, C.; Lieberman, J. A.; Oliveira, T. Y.; Yang, Z.; Abernathy, M. E.; Huey-Tubman, K. E.; Hurley, A.; Turroja, M.; West, K. A.; Gordon, K.; Millard, K. G.; Ramos, V.; Da Silva, J.; Xu, J.; Colbert, R. A.; Patel, R.; Dizon, J.; Unson-O'Brien, C.; Shimeliovich, I.; Gazumyan, A.; Caskey, M.; Bjorkman, P. J.; Casellas, R.; Hatziioannou, T.; Bieniasz, P. D.; Nussenzweig, M. C. MRNA Vaccine-Elicited Antibodies to SARS-CoV-2 and Circulating Variants. Nature 2021, 592 (7855), 616-622. https://doi.org/10.1038/s41586-021-03324-6.
21. Li, Q.; Nie, J.; Wu, J.; Zhang, L.; Ding, R.; Wang, H.; Zhang, Y.; Li, T.; Liu, S.; Zhang, M.; Zhao, C.; Liu, H.; Nie, L.; Qin, H.; Wang, M.; Lu, Q.; Li, X.; Liu, J.; Liang, H.; Shi, Y.; Shen, Y.; Xie, L.; Zhang, L.; Qu, X.; Xu, W.; Huang, W.; Wang, Y. SARS-CoV-2 501Y.V2 Variants Lack Higher Infectivity but Do Have Immune Escape. Cell 2021, 184 (9), 2362-2371.e9. https:/doi.org/10.1016/i.cell.2021.02.042.
22. London School of Hygiene & Tropical Medicine (2021) COVID-19 Vaccine Tracker. Https://Vac-Lshtm.Shinyapps.Io/Ncov_vaccine_landscape/. (Accessed on 30 Dec. 2021).
23. Zhang, Y. N.; Paynter, J.; Sou, C.; Fourfouris, T.; Wang, Y.; Abraham, C.; Ngo, T.; Zhang, Y.; He, L.; Zhu, J. Mechanism of a COVID-19 Nanoparticle Vaccine Candidate That Elicits a Broadly Neutralizing Antibody Response to SARS-CoV-2 Variants. Sci. Adv. 2021, 7 (43), 1-21. https://doi.org/10.1126/sciadv.abj3107.
24. Williams, T. C.; Burgers, W. A. SARS-CoV-2 Evolution and Vaccines: Cause for Concern? Lancet Respir. Med. 2021, 9 (4), 333-335. https://doi.org/10.1016/S2213-2600(21)00075-8.
25. Logunov, D. Y.; Dolzhikova, I. V.; Shcheblyakov, D. V.; Tukhvatulin, A. I.; Zubkova, O. V.; Dzharullaeva, A. S.; Kovyrshina, A. V.; Lubenets, N. L.; Grousova, D. M.; Erokhova, A. S.; Botikov, A. G.; Izhaeva, F. M.; Popova, O.; Ozharovskaya, T. A.; Esmagambetov, I B.; Favorskaya, I. A.; Zrelkin, D. I.; Voronina, D. V.; Shcherbinin, D. N.; Semikhin, A. S.; Simakova, Y. V.; Tokarskaya, E. A.; Egorova, D. A.; Shmarov, M. M.; Nikitenko, N. A.; Gushchin, V. A.; Smolyarchuk, E. A.; Zyryanov, S. K.; Borisevich, S. V.; Naroditsky, B. S.; Gintsburg, A. L. Safety and Efficacy of an RAd26 and RAd5 Vector-Based Heterologous Prime-Boost COVID-19 Vaccine: An Interim Analysis of a Randomised Controlled Phase 3 Trial in Russia. Lancet 2021, 397 (10275), 671-681. https://doi.org/10.1016/S0140-6736(21)00234-8.
26. Heath, P. T.; Galiza, E. P.; Baxter, D. N.; Boffito, M.; Browne, D.; Burns, F.; Chadwick, D. R.; Clark, R.; Cosgrove, C.; Galloway, J.; Goodman, A. L.; Heer, A.; Higham, A.; Iyengar, S.; Jamal, A.; Jeanes, C.; Kalra, P. A.; Kyriakidou, C.; McAuley, D. F.; Meyrick, A.; Minassian, A. M.; Minton, J.; Moore, P.; Munsoor, I.; Nicholls, H.; Osanlou, O.; Packham, J.; Pretswell, C. H.; San Francisco Ramos, A.; Saralaya, D.; Sheridan, R. P.; Smith, R.; Soiza, R. L.; Swift, P. A.; Thomson, E. C.; Turner, J.; Viljoen, M. E.; Albert, G.; Cho, I.; Dubovsky, F.; Glenn, G.; Rivers, J.; Robertson, A.; Smith, K.; Toback, S. Safety and Efficacy of NVX-CoV2373 Covid-19 Vaccine. N. Engl. J. Med. 2021, 385 (13), 1172-1183. https://doi.org/10.1056/nejmoa2107659.
27. Thompson, M. G.; Burgess, J. L.; Naleway, A. L.; Tyner, H.; Yoon, S. K.; Meece, J.; Olsho, L. E. W.; Caban-Martinez, A. J.; Fowlkes, A. L.; Lutrick, K.; Groom, H. C.; Dunnigan, K.; Odean, M. J.; Hegmann, K.; Stefanski, E.; Edwards, L. J.; Schaefer-Solle, N.; Grant, L.; Ellingson, K.; Kuntz, J. L.; Zunie, T.; Thiese, M. S.; Ivacic, L.; Wesley, M. G.; Mayo Lamberte, J.; Sun, X.; Smith, M. E.; Phillips, A. L.; Groover, K. D.; Yoo, Y. M.; Gerald, J.; Brown, R. T.; Herring, M. K.; Joseph, G.; Beitel, S.; Morrill, T. C.; Mak, J.; Rivers, P.; Poe, B. P.; Lynch, B.; Zhou, Y.; Zhang, J.; Kelleher, A.; Li, Y.; Dickerson, M.; Hanson, E.; Guenther, K.; Tong, S.; Bateman, A.; Reisdorf, E.; Barnes, J.; Azziz-Baumgartner, E.; Hunt, D. R.; Arvay, M. L.; Kutty, P.; Fry, A. M.; Gaglani, M. Prevention and Attenuation of Covid-19 with the BNT162b2 and MRNA-1273 Vaccines. N. Engl. J. Med. 2021, 385 (4), 320-329. https://doi.org/10.1056/nejmoa2107058.
28. Baden, L. R.; El Sahly, H. M.; Essink, B.; Kotloff, K.; Frey, S.; Novak, R.; Diemert, D.; Spector, S. A.; Rouphael, N.; Creech, C. B.; McGettigan, J.; Khetan, S.; Segall, N.; Solis, J.; Brosz, A.; Fierro, C.; Schwartz, H.; Neuzil, K.; Corey, L.; Gilbert, P.; Janes, H.; Follmann, D.; Marovich, M.; Mascola, J.; Polakowski, L.; Ledgerwood, J.; Graham, B. S.; Bennett, H.; Pajon, R.; Knightly, C.; Leav, B.; Deng, W.; Zhou, H.; Han, S.; Ivarsson, M.; Miller, J.; Zaks, T. Efficacy and Safety of the MRNA-1273 SARS-CoV-2 Vaccine. N. Engl. J. Med. 2021, 384 (5), 403-416. https://doi.org/10.1056/nejmoa2035389.
29. Lupala, C. S.; Kumar, V.; Su, X. dong; Wu, C.; Liu, H. Computational Insights into Differential Interaction of Mammalian Angiotensin-Converting Enzyme 2 with the SARS-CoV-2 Spike Receptor Binding Domain. Comput. Biol. Med. 2021, 141, 105017. https://doi.org/10.1016/j.compbiomed.2021.105017.
30. Garcia-Beltran, W. F.; Lam, E. C.; St. Denis, K.; Nitido, A. D.; Garcia, Z. H.; Hauser, B. M.; Feldman, J.; Pavlovic, M. N.; Gregory, D. J.; Poznansky, M. C.; Sigal, A.; Schmidt, A. G.; Iafrate, A. J.; Naranbhai, V.; Balazs, A. B. Multiple SARS-CoV-2 Variants Escape Neutralization by Vaccine-Induced Humoral Immunity. Cell 2021, 184 (9), 2523. https://doi.org/10.1016/j.cell.2021.04.006.
31. Wang, P.; Nair, M. S.; Liu, L.; Iketani, S.; Luo, Y.; Guo, Y.; Wang, M.; Yu, J.; Zhang, B.; Kwong, P. D.; Graham, B. S.; Mascola, J. R.; Chang, J. Y.; Yin, M. T.; Sobieszczyk, M.; Kyratsous, C. A.; Shapiro, L.; Sheng, Z.; Huang, Y.; Ho, D. D. Antibody Resistance of SARS-CoV-2 Variants B.1.351 and B.1.1.7. Nature 2021, 593 (7857), 130-135. https://doi.org/10.1038/s41586-021-03398-2.
32. Andreano, E.; Piccini, G.; Licastro, D.; Casalino, L.; Johnson, N. V.; Paciello, I.; Monego, S. D.; Pantano, E.; Manganaro, N.; Manenti, A.; Manna, R.; Casa, E.; Hyseni, I.; Benincasa, L.; Montomoli, E.; Amaro, R. E.; McLellan, J. S.; Rappuoli, R. SARS-CoV-2 Escape from a Highly Neutralizing COVID-19 Convalescent Plasma. Proc. Natl. Acad. Sci. U.S.A 2021, 118 (36), 1-7. https://doi.org/10.1073/pnas.2103154118.
33. Wibmer, C. K.; Ayres, F.; Hermanus, T.; Madzivhandila, M.; Kgagudi, P.; Oosthuysen, B.; Lambson, B. E.; de Oliveira, T.; Vermeulen, M.; van der Berg, K.; Rossouw, T.; Boswell, M.; Ueckermann, V.; Meiring, S.; von Gottberg, A.; Cohen, C.; Morris, L.; Bhiman, J. N.; Moore, P. L. SARS-CoV-2 501Y.V2 Escapes Neutralization by South African COVID-19 Donor Plasma. Nat. Med. 2021, 27 (4), 622-625. https://doi.org/10.1038/s41591-021-01285-x.
34. Cherian, S.; Potdar, V.; Jadhav, S.; Yadav, P.; Gupta, N.; Das, M.; Rakshit, P.; Singh, S.; Abraham, P.; Panda, S.; Team, N. I. C. SARS-CoV-2 Spike Mutations, L452R, T478K, E484Q and P681R, in the Second Wave of COVID-19 in Maharashtra, India. Microorganism 2021, 2, 1-11.
35. Tegally, H.; Wilkinson, E.; Giovanetti, M.; Iranzadeh, A.; Fonseca, V.; Giandhari, J.; Doolabh, D.; Pillay, S.; San, E. J.; Msomi, N.; Mlisana, K.; von Gottberg, A.; Walaza, S.; Allam, M.; Ismail, A.; Mohale, T.; Glass, A. J.; Engelbrecht, S.; Van Zyl, G.; Preiser, W.; Petruccione, F.; Sigal, A.; Hardie, D.; Marais, G.; Hsiao, N. yuan; Korsman, S.; Davies, M. A.; Tyers, L.; Mudau, I.; York, D.; Maslo, C.; Goedhals, D.; Abrahams, S.; Laguda-Akingba, O.; Alisoltani-Dehkordi, A.; Godzik, A.; Wibmer, C. K.; Sewell, B. T.; Lourengo, J.; Alcantara, L. C. J.; Kosakovsky Pond, S. L.; Weaver, S.; Martin, D.; Lessells, R. J.; Bhiman, J. N.; Williamson, C.; de Oliveira, T. Detection of a SARS-CoV-2 Variant of Concern in South Africa. Nature 2021, 592 (7854), 438-443. https://doi.org/10.1038/s41586-021-03402-9.
36. Li, Q.; Wu, J.; Nie, J.; Zhang, L.; Hao, H.; Liu, S.; Zhao, C.; Zhang, Q.; Liu, H.; Nie, L.; Qin, H.; Wang, M.; Lu, Q.; Li, X.; Sun, Q.; Liu, J.; Zhang, L.; Li, X.; Huang, W.; Wang, Y. The Impact of Mutations in SARS-CoV-2 Spike on Viral Infectivity and Antigenicity. Cell 2020, 182 (5), 1284-1294.e9. https://doi.org/10.1016/j.cell.2020.07.012.
37. Rees-Spear, C.; Muir, L.; Griffith, S. A.; Heaney, J.; Aldon, Y.; Snitselaar, J. L.; Thomas, P.; Graham, C.; Seow, J.; Lee, N.; Rosa, A.; Roustan, C.; Houlihan, C. F.; Sanders, R. W.; Gupta, R. K.; Cherepanov, P.; Stauss, H. J.; Nastouli, E.; Doores, K. J.; van Gils, M. J.; McCoy, L. E. The Effect of Spike Mutations on SARS-CoV-2 Neutralization. Cell Rep. 2021, 34 (12), 108890. https://doi.org/10.1016/j.celrep.2021.108890.
38. Jiang, S.; Hillyer, C.; Du, L. Neutralizing Antibodies against SARS-CoV-2 and Other Human Coronaviruses. Trends Immunol. 2020, 41 (5), 355-359. https://doi.org/10.1016/j.it.2020.03.007.
39. Ortega-Rivera, O. A.; Shin, M. D.; Chen, A.; Beiss, V.; Moreno-Gonzalez, M. A.; Lopez-Ramirez, M. A.; Reynoso, M.; Wang, H.; Hurst, B. L.; Wang, J.; Pokorski, J. K.; Steinmetz, N. F. Trivalent Subunit Vaccine Candidates for COVID-19 and Their Delivery Devices. J. Am. Chem. Soc. 2021, 143 (36), 14748-14765. https://doi.org/10.1021/jacs.1c06600.
40. Bachmann, M. F.; Jennings, G. T. Vaccine Delivery: A Matter of Size, Geometry, Kinetics and Molecular Patterns. Nat. Rev. Immunol. 2010, 10 (11), 787-796. https://doi.org/10.1038/nri2868.
41. Shukla, S.; Myers, J. T.; Woods, S. E.; Gong, X.; Czapar, A. E.; Commandeur, U.; Huang, A. Y.; Levine, A. D.; Steinmetz, N. F. Plant Viral Nanoparticles-Based HER2 Vaccine: Immune Response Influenced by Differential Transport, Localization and Cellular Interactions of Particulate Carriers. Biomaterials 2017, 121 (2017), 15-27. https://doi.org/10.1016/j.biomaterials.2016.12.030.
42. Huynh, N. A. T.; Hesketh, E. A. L.; Saxena, P.; Meshcheriakova, Y.; Ku, Y. C.; Hoang, L. A. T.; Johnson, J. A. E.; Ranson, N. A. A.; Lomonossoff, G. A. P.; Reddy, V. A. S. Crystal Structure and Proteomics Analysis of Empty Virus-like Particles of Cowpea Mosaic Virus. Structure 2016, 24 (4), 567-575. https://doi.org/10.1016/j.str.2016.02.011.
43. Ortega-Rivera, O. A.; Shukla, S.; Shin, M. D.; Chen, A.; Beiss, V.; Moreno-Gonzalez, M. A.; Zheng, Y.; Clark, A. E.; Carlin, A. F.; Pokorski, J. K.; Steinmetz, N. F. Cowpea Mosaic Virus Nanoparticle Vaccine Candidates Displaying Peptide Epitopes Can Neutralize the Severe Acute Respiratory Syndrome Coronavirus. ACS Infect. Dis. 2021, 7 (11), 3096-3110. https://doi.org/10.1021/acsinfecdis.1c00410.
44. Wang, C.; Beiss, V.; Steinmetz, N. F. Cowpea Mosaic Virus Nanoparticles and Empty Virus-Like Particles Show Distinct but Overlapping Immunostimulatory Properties. J. Virol. 2019, 93 (21), e00129-19. https://doi.org/10.1128/jvi.00129-19.
45. Murray, A. A.; Wang, C.; Fiering, S.; Steinmetz, N. F. In Situ Vaccination with Cowpea vs Tobacco Mosaic Virus against Melanoma. Mol. Pharm. 2018, 15, 3700-3716. https://doi.org/10.1021/acs.molpharmaceut.8b00316.
46. Shukla, S.; Wang, C.; Beiss, V.; Steinmetz, N. F. Antibody Response against Cowpea Mosaic Viral Nanoparticles Improves in Situ Vaccine Efficacy in Ovarian Cancer. ACS Nano 2020, 14 (3), 2994-3003. https://doi.org/10.1021/acsnano.9b07865.
47. Lizotte, P. H.; Wen, A. M.; Sheen, M. R.; Fields, J.; Rojanasopondist, P.; Steinmetz, N. F. In Situ Vaccination with Cowpea Mosaic Virus Nanoparticles Suppresses Metastatic Cancer. Nat. Nanotechnol. 2015, 11(3), 295-303. https://doi.org/10.1038/nnano.2015.292.
48. Hoopes, P. J.; Wagner, R. J.; Duval, K.; Kang, K.; Gladstone, D. J.; Moodie, K. L.; Crary-Burney, M.; Ariaspulido, H.; Veliz, F. A.; Steinmetz, N. F.; Fiering, S. N. Treatment of Canine Oral Melanoma with Nanotechnology-Based Immunotherapy and Radiation. Mol. Pharm. 2018, 15 (9), 3717-3722. https://doi.org/10.1021/acs.molpharmaceut.8b00126.
49. Zheng, Y.; Lee, P. W.; Wang, C.; Thomas, L. D.; Stewart, P. L.; Steinmetz, N. F.; Pokorski, J. K. Freeze-Drying to Produce Efficacious CPMV Virus-like Particles. Nano Lett. 2019, 19 (3), 2099-2105. https://doi.org/10.1021/acs.nanolett.9b00300.
50. Chenite, A.; Buschmann, M.; Wang, D.; Chaput, C.; Kandani, N. Rheological Characterisation of Thermogelling Chitosan/Glycerol-Phosphate Solutions. Carbohydr. Polym. 2001, 46 (1), 39-47. https://doi.org/10.1016/S0144-8617(00)00281-2.
51. Dash, M.; Chiellini, F.; Ottenbrite, R. M.; Chiellini, E. Chitosan—A Versatile Semi-Synthetic Polymer in Biomedical Applications. Prog. Polym. Sci. 2011, 36 (8), 981-1014. https://doi.org/10.1016/j.progpolymsci.2011.02.001.
52. Lehr, C. M.; Bouwstra, J. A.; Schacht, E. H.; Junginger, H. E. In Vitro Evaluation of Mucoadhesive Properties of Chitosan and Some Other Natural Polymers. Int. J. Pharm. 1992, 78 (1-3), 43-48. https://doi.org/10.1016/0378-5173(92)90353-4.
53. SmithNephew, CarGel BioScaffold Https://Www.Smith-Nephew.Com/Key-Products/Sports-Medicine/Bst-Cargel/; Accessed on 3 Dec. 2021.
54. Illum, L.; Jabbal-Gill, I.; Hinchcliffe, M.; Fisher, A. N.; Davis, S. S. Chitosan as a Novel Nasal Delivery System for Vaccines. Adv. Drug Deliv. Rev. 2001, 51 (1-3), 81-96. https://doi.org/10.1016/S0169-409X(01)00171-5.
55. Ghendon, Y.; Markushin, S.; Krivtsov, G.; Akopova, I. Chitosan as an Adjuvant for Parenterally Administered Inactivated Influenza Vaccines. Arch. Virol. 2008, 153 (5), 831-837. https://doi.org/10.1007/s00705-008-0047-4.
56. ScherlieB, R.; Buske, S.; Young, K.; Weber, B.; Rades, T.; Hook, S. In Vivo Evaluation of Chitosan as an Adjuvant in Subcutaneous Vaccine Formulations. Vaccine 2013, 31 (42), 4812-4819. https://doi.org/10.1016/j.vaccine.2013.07.081.
57. Chenite, A.; Buschmann, M.; Wang, D.; Chaput, C., Kandani, N. Rheological Characterisation of Thermogelling Chitosan/Glycerol-Phosphate Solutions. Carbohydr. Polym. 2001, 46, 39-47.
58. Cho, J.; Heuzey, M. C.; Begin, A.; Carreau, P. J. Physical Gelation of Chitosan in the Presence of β-Glycerophosphate: The Effect of Temperature. Biomacromolecules 2005, 6(6), 3267-3275. https://doi.org/10.1021/bm050313s.
59. Zhou, H. Y.; Jiang, L. J.; Cao, P. P.; Li, J. B.; Chen, X. G. Glycerophosphate-Based Chitosan Thermosensitive Hydrogels and Their Biomedical Applications. Carbohydr. Polym. 2015, 117, 524-536. https://doi.org/10.1016/j.carbpol.2014.09.094.
60. Rafael, D.; Melendres, M. M. R.; Andrade, F.; Montero, S.; Martinez-Trucharte, F.; Vilar-Hernandez, M.; Durin-Lara, E. F.; Schwartz, S.; Abasolo, I. Thermo-Responsive Hydrogels for Cancer Local Therapy: Challenges and State-of-Art. Int. J. Pharm. 2021, 606, 120954. https://doi.org/10.1016/j.ijpharm.2021.120954.
61. Rahmanian-Devin, P.; Baradaran Rahimi, V.; Askari, V. R. Thermosensitive Chitosan-β-Glycerophosphate Hydrogels as Targeted Drug Delivery Systems: An Overview on Preparation and Their Applications. Adv. Pharmacol. Pharm. Sci. 2021, 2021, 6640893. https://doi.org/10.1155/2021/6640893.
62. Al Sabbagh, C.; Seguin, J.; Agapova, E.; Kramerich, D.; Boudy, V.; Mignet, N. Thermosensitive Hydrogels for Local Delivery of 5-Fluorouracil as Neoadjuvant or Adjuvant Therapy in Colorectal Cancer. Eur. J. Pharm. Biopharm. 2020, 157 (October), 154-164. https://doi.org/10.1016/j.ejpb.2020.10.011.
63. Mei, E.; Chen, C.; Li, C.; Ding, X.; Chen, J.; Xi, Q.; Zhou, S.; Liu, J.; Li, Z. Injectable and Biodegradable Chitosan Hydrogel-Based Drug Depot Contributes to Synergistic Treatment of Tumors. Biomacromolecules 2021, 22(12), 5339-5348. https://doi.org/10.1021/acs.biomac.1c01279.
64. Song, K.; Li, liying; Yan, X.; Zhang, W.; Zhang, Y.; Wang, Y.; Liu, T. Characterization of Human Adipose Tissue-Derived Stem Cells in Vitro Culture and in Vivo Differentiation in a Temperature-Sensitive Chitosan/β-Glycerophosphate/Collagen Hybrid Hydrogel. Mater. Sci. Eng. C 2017, 70, 231-240. https://doi.org/10.1016/j.msec.2016.08.085.
65. Stanzione, A.; Polini, A.; La Pesa, V.; Quattrini, A.; Romano, A.; Gigli, G.; Moroni, L.; Gervaso, F. Thermosensitive Chitosan-Based Hydrogels Supporting Motor Neuron-like NSC-34 Cell Differentiation. Biomater. Sci. 2021, 9 (22), 7492-7503. https://doi.org/10.1039/d1bm01129d.
66. Peng, Q.; Sun, X.; Gong, T.; Wu, C. Y.; Zhang, T.; Tan, J.; Zhang, Z. R. Injectable and Biodegradable Thermosensitive Hydrogels Loaded with PHBHHx Nanoparticles for the Sustained and Controlled Release of Insulin. Acta Biomater. 2013, 9 (2), 5063-5069. https://doi.org/10.1016/j.actbio.2012.09.034.
67. Li, X. Y.; Zheng, X. L.; Wei, X. W.; Guo, G.; Gou, M.; Gong, C. Y.; Wang, X. H.; Dai, M.; Chen, L. J.; Wei, Y. Q.; Qian, Z. Y. A Novel Composite Drug Delivery System: Honokiol Nanoparticles in Thermosensitive Hydrogel Based on Chitosan. J. Nanosci. Nanotechnol. 2009, 9 (8), 4586-4592. https://doi.org/10.1166/inn.2009.217.
68. Wen, A. M.; Shukla, S.; Saxena, P.; Aljabali, A. A. A.; Yildiz, I.; Dey, S.; Mealy, J. E.; Yang, A. C.; Evans, D. J.; Lomonossoff, G. P.; Steinmetz, N. F. Interior Engineering of a Viral Nanoparticle and Its Tumor Homing Properties. Biomacromolecules 2012, 13 (12), 3990-4001. https://doi.org/10.1021/bm301278f.
69. Wen, A. M.; Lee, K. L.; Yildiz, I.; Bruckman, M. A.; Shukla, S.; Steinmetz, N. F. Viral Nanoparticles for in Vivo Tumor Imaging. J. Vis. Exp. 2012, 2012, 69, e4352. https://doi.org/10.3791/4352.
70. Shin, M. D.; Hochberg, J. D.; Pokorski, J. K.; Steinmetz, N. F. Bioconjugation of Active Ingredients to Plant Viral Nanoparticles Is Enhanced by Preincubation with a Pluronic F127 Polymer Scaffold. ACS Appl. Mater. Interfaces 2021, 13(50), 59618-59632. https://doi.org/10.1021/acsami.1c13183.
71. Zhou, P.; Li, X.; Zhang, B.; Shi, Q.; Li, D.; Ju, X. A Human Umbilical Cord Mesenchymal Stem Cell-Conditioned Medium/Chitosan/Collagen/β-Glycerophosphate Thermosensitive Hydrogel Promotes Burn Injury Healing in Mice. Biomed Res. Int. 2019, 2019, 5768285. https://doi.org/10.1155/2019/5768285.
72. Wen, A. M.; Infusino, M.; Luca, A. De; Kernan, D. L.; Czapar, A. E.; Strangi, G.; Steinmetz, N. F. Interface of Physics and Biology: Engineering Virus-Based Nanoparticles for Biophotonics. Bioconjugate Chem. 2015, 26, 51-62. https://doi.org/10.1021/bc500524f.
73. Chenite, A.; Chaput, C.; Wang, D.; Combes, C.; Buschmann, M. D.; Hoemann, C. D.; Leroux, J. C.; Atkinson, B. L.; Binette, F.; Selmani, A. Novel Injectable Neutral Solutions of Chitosan Form Biodegradable Gels in Situ. Biomaterials 2000, 21 (21), 2155-2161. https://doi.org/10.1016/S0142-9612(00)00116-2.
74. Liu, P.; Li, M.; Yu, H.; Fang, H.; Yin, J.; Zhu, D.; Yang, Q.; Ke, Q.; Huang, Y.; Guo, Y.; Gao, Y.; Zhang, C. Biphasic CK2.1-Coated β-Glycerophosphate Chitosan/LL37-Modified Layered Double Hydroxide Chitosan Composite Scaffolds Enhance Coordinated Hyaline Cartilage and Subchondral Bone Regeneration. Chem. Eng. J. 2021, 418 (December 2020), 129531. https://doi.org/10.1016/j.cej.2021.129531.
75. Aliaghaei, M.; Mirzadeh, H.; Dashtimoghadam, E.; Taranejoo, S. Investigation of Gelation Mechanism of an Injectable Hydrogel Based on Chitosan by Rheological Measurements for a Drug Delivery Application. Soft Matter 2012, 8 (27), 7128-7137. https://doi.org/10.1039/c2sm25254f.
76. Teles, H.; Vermonden, T.; Eggink, G.; Hennink, W. E.; de Wolf, F. A. Hydrogels of Collagen-Inspired Telechelic Triblock Copolymers for the Sustained Release of Proteins. J. Control. Release 2010, 147 (2), 298-303. https://doi.org/10.1016/j.jconrel.2010.07.098.
77. Ukmar, T.; Maver, U.; Planinsek, O.; Kaucic, V.; Gaberscek, M.; Godec, A. Understanding Controlled Drug Release from Mesoporous Silicates: Theory and Experiment. J. Control. Release 2011, 155 (3), 409-417. https://doi.org/10.1016/j.jconrel.2011.06.038.
78. Ukmar, T.; Gaberek, M.; Merzel, F.; Godec, A. Modus Operandi of Controlled Release from Mesoporous Matrices: A Theoretical Perspective. Phys. Chem. Chem. Phys. 2011, 13 (33), 15311-15317. https://doi.org/10.1039/c1cp20636b.
79. Czapar, A. E.; Tiu, B. D. B.; Veliz, F. A.; Pokorski, J. K.; Steinmetz, N. F. Slow-Release Formulation of Cowpea Mosaic Virus for In Situ Vaccine Delivery to Treat Ovarian Cancer. Adv. Sci. 2018, 5 (5), 1-8. https://doi.org/10.1002/advs.201700991.
80. Cai, H.; Shukla, S.; Wang, C.; Masarapu, H.; Steinmetz, N. F. Heterologous Prime-Boost Enhances the Antitumor Immune Response Elicited by Plant-Virus-Based Cancer Vaccine. J. Am. Chem. Soc. 2019, 141 (16), 6509-6518. https://doi.org/10.1021/jacs.9b01523.
81. Shukla, S.; Jandzinski, M.; Wang, C.; Gong, X.; Bonk, K. W.; Keri, R. A.; Steinmetz, N. F. A Viral Nanoparticle Cancer Vaccine Delays Tumor Progression and Prolongs Survival in a HER2+ Tumor Mouse Model. Adv. Ther. 2019, 2 (4), 1800139. https://doi.org/10.1002/adtp.201800139.
82. Smith, A.; Perelman, M.; Hinchcliffe, M. Chitosan a Promising Safe and Immune-Enhancing Adjuvant for Intranasal Vaccines. Hum. Vaccines Immunother. 2014, 10 (3), 797-807. https://doi.org/10.4161/hv.27449.
83. Mohsen, M. O.; Augusto, G.; Bachmann, M. F. The 3Ds in Virus-like Particle Based-Vaccines: “Design, Delivery and Dynamics.” Immunol. Rev. 2020, 296 (1), 155-168. https://doi.org/10.1111/imr.12863.
84. Zellweger, R. M.; Wartel, T. A.; Marks, F.; Song, M.; Kim, J. H. Vaccination against SARS-CoV-2 and Disease Enhancement—Knowns and Unknowns. Expert Rev. Vaccines 2020, 19 (8), 691-698. https://doi.org/10.1080/14760584.2020.1800463.
85. Munoz, F. M.; Cramer, J. P.; Dekker, C. L.; Dudley, M. Z.; Graham, B. S.; Gurwith, M.; Law, B.; Perlman, S.; Polack, F. P.; Spergel, J. M.; Braeckel, E. Van; Ward, B. J.; Didierlaurent, A. M.; Henri, P. Vaccine-Associated Enhanced Disease: Case Definition and Guidelines for Data Collection, Analysis, and Presentation of Immunization Safety Data. Vaccine 2021, 39 (22), 3053-3066. https://doi.org/10.1016/j.vaccine.2021.01.055.
86. Lee, W. S.; Wheatley, A. K.; Kent, S. J.; DeKosky, B. J. Antibody-Dependent Enhancement and SARS-CoV-2 Vaccines and Therapies. Nat. Microbiol. 2020, 5 (10), 1185-1191. https://doi.org/10.1038/s41564-020-00789-5.
87. Hotez, P. J.; Corry, D. B.; Bottazzi, M. E. COVID-19 Vaccine Design: The Janus Face of Immune Enhancement. Nat. Rev. Immunol. 2020, 20 (6), 347-348. https://doi.org/10.1038/s41577-020-0323-4.
88. Al-Saraireh, Y. M.; Alshammari, F. O. F. O.; Youssef, A. M. M.; Al-Sarayreh, S.; Almuhaisen, G. H.; Alnawaiseh, N.; Al-Shuneigat, J. M.; Alrawashdeh, H. M. Cytochrome 4z1 Expression Is Associated with Poor Prognosis in Colon Cancer Patients. Onco. Targets. Ther. 2021, 14, 5249-5260. https://doi.org/10.2147/OT T.S332037.
89. Cancer—WHO, World Health Organization 2022. Https://Www.Who.Int/News-Room/Fact-Sheets/Detail/Cancer (Accessed 29 Jan. 2022).
90. Siegel, R. L.; Miller, K. D.; Fuchs, H. E.; Jemal, A. Cancer Statistics, 2021. CA. Cancer J. Clin. 2021, 71 (1), 7-33. https://doi.org/10.3322/caac.21654.
91. Torre, L. A.; Bray, F.; Siegel, R. L.; Ferlay, J.; Lortet-Tieulent, J.; Jemal, A. Global Cancer Statistics, 2012. CA. Cancer J. Clin. 2015, 65 (2), 87-108. https://doi.org/10.3322/caac.21262.
92. Kuipers, E. J.; Grady, W. M.; Lieberman, D.; Seufferlein, T.; Sung, J. J.; Boelens, P. G.; Van De Velde, C. J. H.; Watanabe, T. Colorectal Cancer. Nat. Rev. Dis. Prim. 2015, 1, 1-25. https://doi.org/10.1038/nrdp.2015.65.
93. Luo, D.; Yang, Y.; Shan, Z.; Liu, Q.; Cai, S.; Li, Q.; Li, X. Clinicopathological Features of Stage I-III Colorectal Cancer Recurrence Over 5 Years After Radical Surgery Without Receiving Neoadjuvant Therapy: Evidence From a Large Sample Study. Front. Surg. 2021, 8 (August), 1-6. https://doi.org/10.3389/fsurg.2021.666400.
94. You, B.; Chen, E. X. Anti-EGFR Monoclonal Antibodies for Treatment of Colorectal Cancers: Development of Cetuximab and Panitumumab. J. Clin. Pharmacol. 2012, 52 (2), 128-155. https://doi.org/10.1177/0091270010395940.
95. Mao, C.; Beiss, V.; Fields, J.; Steinmetz, N. F.; Fiering, S. Cowpea Mosaic Virus Stimulates Antitumor Immunity through Recognition by Multiple MYD88-Dependent Toll-like Receptors. Biomaterials 2022, 275 (120914). https://doi.org/10.1016/j.biomaterials.2021.120914.Cowpea.
96. Koellhoffer, E. C.; Mao, C.; Beiss, V.; Wang, L.; Fiering, S. N.; Boone, C. E.; Steinmetz, N. F. Inactivated Cowpea Mosaic Virus in Combination with OX40 Agonist Primes Potent Antitumor Immunity in a Bilateral Melanoma Mouse Model. Mol. Pharm. 2021. https://doi.org/10.1021/acs.molpharmaceut.1c00681.
97. Lizotte, P. H.; Wen, A. M.; Sheen, M. R.; Fields, J.; Rojanasopondist, P.; Steinmetz, N. F.; Fiering, S. In Situ Vaccination with Cowpea Mosaic Virus Nanoparticles Suppresses Metastatic Cancer. Nat. Nanotechnol. 2016, 11 (3), 295-303. https://doi.org/10.1038/nnano.2015.292.
98. Shukla, S.; Myers, J. T.; Woods, S. E.; Gong, X.; Czapar, A. E.; Commandeur, U.; Huang, A. Y.; Levine, A. D.; Steinmetz, N. F. Plant Viral Nanoparticles-Based HER2 Vaccine: Immune Response Influenced by Differential Transport, Localization and Cellular Interactions of Particulate Carriers. Biomaterials 2017, 121 (2017), 15-27. https://doi.org/10.1016/j.biomaterials.2016.12.030.
99. Shukla, S.; Wang, C.; Beiss, V.; Steinmetz, N. F. Antibody Response against Cowpea Mosaic Viral Nanoparticles Improves in Situ Vaccine Efficacy in Ovarian Cancer. ACS Nano 2020, 14 (3), 2994-3003. https://doi.org/10.1021/acsnano.9b07865.
100. Hoopes, P. J.; Wagner, R. J.; Duval, K.; Kang, K.; Gladstone, D. J.; Moodie, K. L.; Crary-Burney, M.; Ariaspulido, H.; Veliz, F. A.; Steinmetz, N. F.; Fiering, S. N. Treatment of Canine Oral Melanoma with Nanotechnology-Based Immunotherapy and Radiation. Mol. Pharm. 2018, 15 (9), 3717-3722. https://doi.org/10.1021/acs.molpharmaceut.8b00126.
101. Chung, Y. H.; Park, J.; Cai, H.; Steinmetz, N. F. S100A9-Targeted Cowpea Mosaic Virus as a Prophylactic and Therapeutic Immunotherapy against Metastatic Breast Cancer and Melanoma. Adv. Sci. 2021, 8 (21), 1-15. https://doi.org/10.1002/advs.202101796.
102. Czapar, A. E.; Tiu, B. D. B.; Veliz, F. A.; Pokorski, J. K.; Steinmetz, N. F. Slow-Release Formulation of Cowpea Mosaic Virus for In Situ Vaccine Delivery to Treat Ovarian Cancer. Adv. Sci. 2018, 5 (5), 1-8. https://doi.org/10.1002/advs.201700991.
103. Wright, A. A.; Cronin, A.; Milne, D. E.; Bookman, M. A.; Burger, R. A.; Cohn, D. E.; Cristea, M. C.; Griggs, J. J.; Keating, N. L.; Levenback, C. F.; Mantia-Smaldone, G.; Matulonis, U. A.; Meyer, L. A.; Niland, J. C.; Weeks, J. C.; O'Malley, D. M. Use and Effectiveness of Intraperitoneal Chemotherapy for Treatment of Ovarian Cancer. J. Clin. Oncol. 2015, 33 (26), 2841-2847. https://doi.org/10.1200/JCO.2015.61.4776. (17)
104. Albertazzi, L.; Gherardini, L.; Brondi, M.; Sulis Sato, S.; Bifone, A.; Pizzorusso, T.; Ratto, G. M.; Bardi, G. In Vivo Distribution and Toxicity of PAMAM Dendrimers in the Central Nervous System Depend on Their Surface Chemistry. Mol. Pharm. 2013, 10 (1), 249-260. https://doi.org/10.1021/mp300391v.
105. Mukherjee, S. P.; Lyng, F. M.; Garcia, A.; Davoren, M.; Byrne, H. J. Mechanistic Studies of in Vitro Cytotoxicity of Poly(Amidoamine) Dendrimers in Mammalian Cells. Toxicol. Appl. Pharmacol. 2010, 248 (3), 259-268. https://doi.org/10.1016/j.taap.2010.08.016.
106. Nkanga, C. I.; Steinmetz, N. F. The Pharmacology of Plant Virus Nanoparticles. Virology 2021, 556, 39-61. https://doi.org/10.1016/j.virol.2021.01.012.
107. Nikitin, N. A.; Trifonova, E. A.; Karpova, O. V.; Atabekov, J. G. Biosafety of Plant Viruses for Human and Animals. Moscow Univ. Biol. Sci. Bull. 2016, 71 (3), 128-134. https://doi.org/10.3103/S0096392516030081.
108. Aranaz, I.; Alcintara, A. R.; Civera, M. C.; Arias, C.; Elorza, B.; Caballero, A. H.; Acosta, N. Chitosan: An Overview of Its Properties and Applications. Polymers (Basel). 2021, 13 (19). https://doi.org/10.3390/polyml3193256.
109. European Pharmacopoeia, 10th Ed.; Council of Europe, European Directorate for the Quality of Medicines Healthcare: Strasbourg, France, 2019.
110. The United States Pharmacopeial Convention. The United States Pharmacopeia: The National Formulary; The United States Pharmacopeial Convention: Rockville, MD, 2018.
111. SmithNephew, CarGel BioScaffold Https://Www.Smith-Nephew.Com/Key-Products/Sports-Medicine/Bst-Cargel/; Accessed on 3 Dec. 2021. 2021.
112. Zhou, H. Y.; Jiang, L. J.; Cao, P. P.; Li, J. B.; Chen, X. G. Glycerophosphate-Based Chitosan Thermosensitive Hydrogels and Their Biomedical Applications. Carbohydr. Polym. 2015, 117, 524-536. https://doi.org/10.1016/j.carbpol.2014.09.094.
113. Cho, J.; Heuzey, M. C.; Begin, A.; Carreau, P. J. Physical Gelation of Chitosan in the Presence of β-Glycerophosphate: The Effect of Temperature. Biomacromolecules 2005, 6 (6), 3267-3275. https://doi.org/10.1021/bm050313s.
114. Mei, E.; Chen, C.; Li, C.; Ding, X.; Chen, J.; Xi, Q.; Zhou, S.; Liu, J.; Li, Z. Injectable and Biodegradable Chitosan Hydrogel-Based Drug Depot Contributes to Synergistic Treatment of Tumors. Biomacromolecules 2021. https://doi.org/10.1021/acs.biomac.1c01279.
115. Song, K.; Li, liying; Yan, X.; Zhang, W.; Zhang, Y.; Wang, Y.; Liu, T. Characterization of Human Adipose Tissue-Derived Stem Cells in Vitro Culture and in Vivo Differentiation in a Temperature-Sensitive Chitosan/β-Glycerophosphate/Collagen Hybrid Hydrogel. Mater. Sci. Eng. C 2017, 70, 231-240. https://doi.org/10.1016/j.msec.2016.08.085.
116. Peng, Q.; Sun, X.; Gong, T.; Wu, C. Y.; Zhang, T.; Tan, J.; Zhang, Z. R. Injectable and Biodegradable Thermosensitive Hydrogels Loaded with PHBHHx Nanoparticles for the Sustained and Controlled Release of Insulin. Acta Biomater. 2013, 9 (2), 5063-5069. https://doi.org/10.1016/j.actbio.2012.09.034.
117. Li, X. Y.; Zheng, X. L.; Wei, X. W.; Guo, G.; Gou, M.; Gong, C. Y.; Wang, X. H.; Dai, M.; Chen, L. J.; Wei, Y. Q.; Qian, Z. Y. A Novel Composite Drug Delivery System: Honokiol Nanoparticles in Thermosensitive Hydrogel Based on Chitosan. J. Nanosci. Nanotechnol. 2009, 9 (8), 4586-4592. https://doi.org/10.1166/jnn.2009.217.
118. Wen, A. M.; Lee, K. L.; Yildiz, I.; Bruckman, M. A.; Shukla, S.; Steinmetz, N. F. Viral Nanoparticles for in Vivo Tumor Imaging. J. Vis. Exp. 2012, No. 69. https://doi.org/10.3791/4352.
119. Wen, A. M.; Shukla, S.; Saxena, P.; Aljabali, A. A. A.; Yildiz, I.; Dey, S.; Mealy, J. E.; Yang, A. C.; Evans, D. J.; Lomonossoff, G. P.; Steinmetz, N. F. Interior Engineering of a Viral Nanoparticle and Its Tumor Homing Properties. Biomacromolecules 2012, 13 (12), 3990-4001. https://doi.org/10.1021/bm301278f.
120. Nkanga, C. I.; Ortega-rivera, O. A.; Shin, M. D.; Miguel, A.; Steinmetz, N. F. Injectable Slow-Release Hydrogel Formulation of a Plant Virus-Based COVID-19 Vaccine Candidate. 2022.
121. Saunders, K.; Sainsbury, F.; Lomonossoff, G. P. Efficient Generation of Cowpea Mosaic Virus Empty Virus-like Particles by the Proteolytic Processing of Precursors in Insect Cells and Plants. Virology 2009, 393 (2), 329-337. https://doi.org/10.1016/j.virol.2009.08.023.
122. Wen, A. M.; Infusino, M.; Luca, A. De; Kernan, D. L.; Czapar, A. E.; Strangi, G.; Steinmetz, N. F. Interface of Physics and Biology: Engineering Virus-Based Nanoparticles for Biophotonics. Bioconjugate Chem. 2015, 26, 51-62. https://doi.org/10.1021/bc500524f.
123. Al Sabbagh, C.; Seguin, J.; Agapova, E.; Kramerich, D.; Boudy, V.; Mignet, N. Thermosensitive Hydrogels for Local Delivery of 5-Fluorouracil as Neoadjuvant or Adjuvant Therapy in Colorectal Cancer.Eur. J. Pharm. Biopharm. 2020, 157 (October), 154-164. https://doi.org/10.1016/j.ejpb.2020.10.011.
124. Stanzione, A.; Polini, A.; La Pesa, V.; Quattrini, A.; Romano, A.; Gigli, G.; Moroni, L.; Gervaso, F. Thermosensitive Chitosan-Based Hydrogels Supporting Motor Neuron-like NSC-34 Cell Differentiation. Biomater. Sci. 2021, 9 (22), 7492-7503. https://doi.org/10.1039/d1bm01129d.
125. Ukmar, T.; Gaberek, M.; Merzel, F.; Godec, A. Modus Operandi of Controlled Release from Mesoporous Matrices: A Theoretical Perspective. Phys. Chem. Chem. Phys. 2011, 13 (33), 15311-15317. https://doi.org/10.1039/c1cp20636b.
126. Ukmar, T.; Maver, U.; Planinšek, O.; Kaučič, V.; Gaberšček, M.; Godec, A. Understanding Controlled Drug Release from Mesoporous Silicates: Theory and Experiment. J. Control. Release 2011, 155 (3), 409-417. https://doi.org/10.1016/j.jconrel.2011.06.038.
127. Marbrook, J.; Matthews, R. E. F. The Differential Immunogenicity of Plant Viral Protein and Nucleoproteins. Virology 1966, 28 (2), 219-228. https://doi.org/10.1016/0042-6822(66)90146-2.
128. Murray, A. A.; Wang, C.; Fiering, S.; Steinmetz, N. F. In Situ Vaccination with Cowpea vs Tobacco Mosaic Virus against Melanoma. Mol. Phar 2018, 15 (9), 3700-3716. https://doi.org/10.1021/acs.molpharmaceut.8b00316.
129. Xu, S.; Fan, H.; Yin, L.; Zhang, J.; Dong, A.; Deng, L.; Tang, H. Thermosensitive Hydrogel System Assembled by PTX-Loaded Copolymer Nanoparticles for Sustained Intraperitoneal Chemotherapy of Peritoneal Carcinomatosis. Eur. J. Pharm. Biopharm. 2016, 104, 251-259. https://doi.org/10.1016/j.ejpb.2016.05.010.
130. Shukla, S.; Ablack, A. L.; Wen, A. M.; Lee, K. L.; Lewis, J. D.; Steinmetz, N. F. Increased Tumor Homing and Tissue Penetration of the Filamentous Plant Viral Nanoparticle Potato Virus X. Mol. Pharm. 2013, 10 (1), 33-42. https://doi.org/10.1021/mp300240m.
131. Singh, P.; Prasuhn, D.; Yeh, R. M.; Destito, G.; Rae, C. S.; Osborn, K.; Finn, M. G.; Manchester, M. Bio-Distribution, Toxicity and Pathology of Cowpea Mosaic Virus Nanoparticles in Vivo. J. Control. Release 2007, 120 (1-2), 41-50. https://doi.org/10.1016/j.jconrel.2007.04.003.
132. Bachmann, M. F.; Jennings, G. T. Vaccine Delivery: A Matter of Size, Geometry, Kinetics and Molecular Patterns. Nat. Rev. Immunol. 2010, 10 (11), 787-796. https://doi.org/10.1038/nri2868.
133. Mohsen, M. O.; Augusto, G.; Bachmann, M. F. The 3Ds in Virus-like Particle Based-Vaccines: “Design, Delivery and Dynamics.” Immunol. Rev. 2020, 296 (1), 155-168. https://doi.org/10.1111/imr.12863.
134. Smyth, J. W. P.; Fleeton, M. N.; Sheahan, B. J.; Atkins, G. J. Treatment of Rapidly Growing K-BALB and CT26 Mouse Tumours Using Semliki Forest Virus and Its Derived Vector. Gene Ther. 2005, 12 (2), 147-159. https://doi.org/10.1038/sj.gt.3302390.
135. Su, Z.; Shi, Y.; Teng, L.; Li, X.; Wang, L.; Meng, Q.; Teng, L.; Li, Y.-X. Biodegradable Poly(D,L-Lactide-Co-Glycolide) (PLGA) Microspheres for Sustained Release of Risperidone: Zero-Order Release Formulation. Pharm. Dev. Technol. 2011, 16 (4), 377-384. https://doi.org/10.3109/10837451003739297.

1. A formulation comprising a virus-like particle (VLP) derived from a plant virus optionally conjugated to a therapeutic peptide, a chitosan polymer and β-glycerophosphate (GP), optionally wherein the chitosan has a molecular weight from about 250 kDa to about 1500 kDa, and further optionally wherein the concentration of the VLP in the formulation comprises from about 0.1 mg/ml to about 7 mg/ml at 37° C.

2-3. (canceled)

4. The formulation of claim 1, wherein the plant virus is from the genus Bromovirus, Comovirus, or Tymovirus, optionally wherein the plant virus is Cowpea chlorotic mottle virus (CCMV), Cowpea mosaic virus (CPMV), or Physalis mottle virus (PhMV).

5. (canceled)

6. The formulation of claim 1, wherein the VLP comprises a capsid protein, optionally a modified capsid protein, further optionally from CCMV, CPMV, PhMV, or a combination thereof.

7-18. (canceled)

19. The formulation of claim 1, comprising a plurality of VLPs, optionally wherein the therapeutic peptides in the plurality are the same or different from each other, and further optionally wherein the VLPs in the plurality are the same or different from each other.

20-21. (canceled)

22. A composition comprising the formulation of claim 1 and a carrier, optionally a pharmaceutically acceptable carrier, and further optionally comprising a preservative or stabilizer.

23-27. (canceled)

28. A method of treating a disease or condition or inducing an immune response in a subject in need thereof, comprising administering to the subject a formulation of claim 1.

29. The method of claim 28, wherein the disease or condition is selected from a cancer, an inflammatory condition, an autoimmune disease, an allergy, or a pathogenic infection and the optional therapeutic peptide is selected to treat or induce an immune response against the disease or condition.

30. The method of claim 28, further comprising administering an effective amount of a VLP derived from a plant virus optionally conjugated to a therapeutic peptide, wherein the therapeutic peptide is selected for efficacy against the disease or condition.

31-36. (canceled)

37. A method for formulating a VLP, comprising admixing a virus-like particle (VLP) derived from a plant virus optionally conjugated to a therapeutic peptide, a chitosan polymer and β-glycerophosphate (GP), optionally wherein the chitosan has a molecular weight from about 250 kDa to about 1500 kDa, and further optionally wherein the concentration of the VLP in the formulation comprises from about 0.1 mg/ml to about 7 mg/ml at 37° C.

38-39. (canceled)

40. The method of claim 37, wherein the plant virus is from the genus Bromovirus, Comovirus, or Tymovirus, and optionally wherein the plant virus is Cowpea chlorotic mottle virus (CCMV), Cowpea mosaic virus (CPMV), or Physalis mottle virus (PhMV).

41. (canceled)

42. The method of claim 37, wherein the VLP comprises a capsid protein, optionally a modified capsid protein, further optionally from CCMV, CPMV, PhMV, or a combination thereof.

43-57. (canceled)

58. A kit comprising the formulation of claim 1, and instructions for use.


SEQUENCE LISTING (19 sequences)

SEQ ID NO: 1 - protein, 18 aa, Severe acute respiratory syndrome coronavirus 2
PSKPSKRSFIEDLLFNKV

SEQ ID NO: 2 - protein, 18 aa, Severe acute respiratory syndrome coronavirus 2
YNSASFSTFKCYGVSPTK

SEQ ID NO: 3 - protein, 15 aa, Severe acute respiratory syndrome coronavirus 2
LPDPSKPSKRSFIED

SEQ ID NO: 4 - protein, 5 aa, Severe acute respiratory syndrome coronavirus 2
FRKSN

SEQ ID NO: 5 - protein, 18 aa, Severe acute respiratory syndrome coronavirus 2
TESNKKFLPFQQFGRDIA

SEQ ID NO: 6 - protein, 12 aa, Severe acute respiratory syndrome coronavirus 2
TESNKKFLPFQQ

SEQ ID NO: 7 - protein, 12 aa, Severe acute respiratory syndrome coronavirus 2
HADQLTPTWRVY

SEQ ID NO: 8 - protein, 12 aa, Severe acute respiratory syndrome coronavirus 2
FKEELDKYFKNH

SEQ ID NO: 9 - protein, 15 aa, Severe acute respiratory syndrome coronavirus 2
FASTEKSNIIRGWIF

SEQ ID NO: 10 - protein, 15 aa, Severe acute respiratory syndrome coronavirus 2
PFLGVYYHKNNKSWM

SEQ ID NO: 11 - protein, 15 aa, Severe acute respiratory syndrome coronavirus 2
EVRQIAPGQTGKIAD

SEQ ID NO: 12 - protein, 16 aa, Severe acute respiratory syndrome coronavirus 2
NNLDSKVGGNYNYLYR

SEQ ID NO: 13 - protein, 15 aa, Severe acute respiratory syndrome coronavirus 2
LFRKSNLKPFERDIS

SEQ ID NO: 14 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 15 - protein, 374 aa, Cowpea mosaic virus
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 16 - protein, 189 aa, Cowpea mosaic virus
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 17 - protein, 188 aa, Physalis mottle virus
MDSSEVVKVKQASIPAPGSILSQPNTEQSPAIVLPFQFEATTFGTAETAAQVSLQTADPITKLTAPYRHAQIVECKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 18 - protein, 268 aa, Sesbania mosaic virus
MAKRLSKQQLAKAIANTLETPPQPKAGRRRNRRRQRSAVQQLQPTQAGISMAPSAQGAMVRIRNPAVSSSRGGITVLTHSELSAEIGVTDSIVVSSELVMPYTVGTWLRGVAANWSKYSWLSVRYTYIPSCPSSTAGSIHMGFQYDMADTVPVSVNQLSNLRGYVSGQVWSGSAGLCFINGTRCSDTSTAISTTLDVSKLGKKWYPYKTSADYATAVGVDVNIATPLVPARLVIALLDGSSSTAVAAGRIYCTYTIQMIEPTASALNN

SEQ ID NO: 19 - protein, 22 aa, synthetic construct
CGGGPSKPSKRSFIEDLLFNKV